(19) United States
(12) Patent Application Publication
(10) Pub. No.: US 2025/0353847 A1
(43) Pub. Date: Nov. 20, 2025

(54) CRYSTALLINE FORMS OF AN AKRIC3 DEPENDENT KARS INHIBITOR

(71) Applicant: Novartis AG, Basel (CH)

(72) Inventors: Danuta LUBICKA, Peabody, MA (US); Michael MUTZ, Freiburg im Breisgau (DE); Yuling ZHU, Shanghai (CN)

(21) Appl. No.: 18/871,590

(22) PCT Filed: Jul. 20, 2023
(86) PCT No.: PCT/IB2023/057425

§ 371 Date: Dec. 4, 2024

(30) Foreign Application Priority Data
Jul. 26, 2022 (WO) PCT/CN2022/107941

(51) Int. Cl. C07D 471/10

(52) U.S. Cl. CPC C07D 471/10

(57) ABSTRACT

This application relates to crystalline forms of an inhibitor of AKR1C3 dependent KARS.

### FIELD OF INVENTION

The present disclosure relates to a crystalline form of 6′-fluoro-N-(4-fluorobenzyl)-4′-oxo-3′,4′-dihydro-1′H-spiro[piperidine-4,2′-quinoline]-1-carboxamide.

The present disclosure also relates to a pharmaceutical composition comprising the crystalline form, as well as methods for obtaining such crystalline form and methods of using such crystalline form in the treatment of diseases and disorders which are typically ameliorated by the inhibition of AKR1C3 dependent KARS. Such diseases and disorders may include cancers with genetic alterations on the NRF2/KEAP1 pathway such as solid tumors, from non-small cell lung cancer (NSCLC), liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), and myelodysplastic syndrome (MDS).

### BACKGROUND

Polymorphism denotes the existence of more than one crystalline form of a substance.

Solid state form of the active pharmaceutical ingredient (API) of a particular drug is often an important determinant of the drug's ease of preparation, hygroscopicity, stability, solubility, storage stability, ease of formulation, rate of dissolution in gastrointestinal fluids and in vivo bioavailability. Crystalline forms occur where the same composition of matter crystallizes in a different lattice arrangement resulting in different thermodynamic properties and stabilities specific to the particular crystalline form. Crystalline forms may also include different hydrates or solvates of the same compound. In deciding which form is preferable, the numerous properties of the forms are compared and the preferred form chosen based on the many physical property variables. It is entirely possible that one form can be preferable in some circumstances where certain aspects such as ease of preparation, stability, etc. are deemed to be critical. In other situations, a different form may be preferred for greater dissolution rate and/or superior bioavailability.

Therefore, this ability of a chemical substance to crystallize in more than one crystalline form can have a profound effect on the shelf life, solubility, formulation properties, and processing properties of a drug. In addition, the action of a drug can be affected by the polymorphism of the drug molecule. Different polymorphs can have different rates of uptake in the body, leading to lower or higher biological activity than desired. In extreme cases, an undesired polymorph can even show toxicity. The occurrence of an unknown crystalline form during manufacture can have a significant impact.

It is not yet possible to predict whether a particular compound or salt of a compound will form polymorphs, whether any such polymorphs will be suitable for commercial use in a therapeutic composition, or which polymorphs will display such desirable properties. However, understanding which crystalline forms of a drug are possible in certain cases allows researchers to maximize the desired properties of a compound, such as solubility, formulation properties, processing properties, and shelf life. Understanding these factors early in the development of a new drug may mean a more active, more stable, or more cheaply manufactured drug.

Therefore, there is a need to provide a solid state form of 6′-fluoro-N-(4-fluorobenzyl)-4′-oxo-3′,4′-dihydro-1′H-spiro[piperidine-4,2′-quinoline]-1-carboxamide, which possesses physicochemical properties allowing for a reliable production of a safe and efficacious drug product comprising 6′-fluoro-N-(4-fluorobenzyl)-4′-oxo-3′,4′-dihydro-1′H-spiro[piperidine-4,2′-quinoline]-1-carboxamide.

Example 40 of WO2021/005586, published Jan. 14, 2021, discloses 6′-fluoro-N-(4-fluorobenzyl)-4′-oxo-3′,4′-dihydro-1′H-spiro[piperidine-4,2′-quinoline]-1-carboxamide.

6′-fluoro-N-(4-fluorobenzyl)-4′-oxo-3′,4′-dihydro-1′H-spiro[piperidine-4,2′-quinoline]-1-carboxamide has the structure of Formula (I):

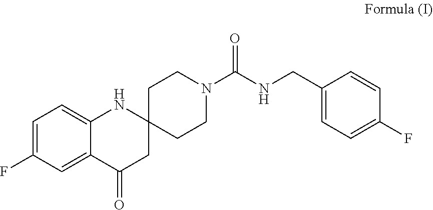 [SMILES: O=C1CC2(CCN(C(=O)NCc3ccc(F)cc3)CC2)Nc2ccc(F)cc21]

6′-fluoro-N-(4-fluorobenzyl)-4′-oxo-3′,4′-dihydro-1′H-spiro[piperidine-4,2′-quinoline]-1-carboxamide is converted to the lysine t-RNA synthetase (KARS) inhibitor (R)-6′-fluoro-N-(4-fluorobenzyl)-4′-hydroxy-3′,4′-dihydro-1′H-spiro[piperidine-4,2′-quinoline]-1-carboxamide by AKR1C3 in the presence of NADPH (the reduced form of nicotinamide adenine dinucleotide phosphate). (R)-6′-fluoro-N-(4-fluorobenzyl)-4′-hydroxy-3′,4′-dihydro-1′H-spiro[piperidine-4,2′-quinoline]-1-carboxamide is disclosed in example 152 of WO2021/005586 and has the structure of Formula (II):

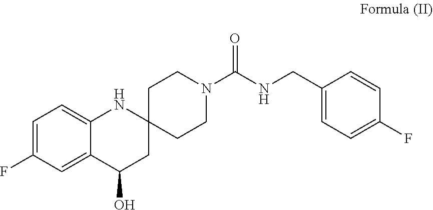 [SMILES: O=C(NCc1ccc(F)cc1)N1CCC2(CC1)C[C@@H](O)c1cc(F)ccc1N2]

Lysine t-RNA synthetase is a ubiquitous enzyme essential for protein synthesis that is part of the multi-tRNA synthetase complex. AKR1C3 (also named type 2 3α(17β)-hydroxysteroid dehydrogenase) is an NADP(H)-dependent ketosteroid reductase, member of the aldo-keto reductase (AKR) superfamily, that plays a role in steroid hormone metabolism and signaling, as well as xenobiotic detoxification.

WO2021/005586, however, provides no information about crystalline forms of 6′-fluoro-N-(4-fluorobenzyl)-4′-oxo-3′,4′-dihydro-1′H-spiro[piperidine-4,2′-quinoline]-1-carboxamide.

A crystalline form of 6′-fluoro-N-(4-fluorobenzyl)-4′-oxo-3′,4′-dihydro-1′H-spiro[piperidine-4,2′-quinoline]-1-carboxamide has been discovered, which is useful in treating diseases which are typically ameliorated by the inhibition of AKR1C3 dependent KARS. Such diseases and conditions include cancers, such as, solid tumors, non-small cell lung cancer (NSCLC), liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), and myelodysplastic syndrome (MDS).

### SUMMARY

In one aspect, the present invention provides a crystalline form of 6′-fluoro-N-(4-fluorobenzyl)-4′-oxo-3′,4′-dihydro-1′H-spiro[piperidine-4,2′-quinoline]-1-carboxamide in a free form (i.e. a non-salt form). In a particular embodiment, the free form is an anhydrous form of the compound of Formula (I). In an embodiment, the crystalline form includes the form designated herein as Form A.

Preferably, crystalline Form A is substantially pure. More preferably, crystalline Form A is substantially phase pure.

The designation “Form A” is a name used herein to identify a specific form, and should not be considered limiting with respect to any other substance possessing similar or identical physical and chemical characteristics, but rather it should be understood that these designations are mere identifiers that should be interpreted according to the characterization information also presented herein.

In one aspect, the present invention also provides a pharmaceutical composition comprising: (a) a therapeutically effective amount of crystalline Form A of 6′-fluoro-N-(4-fluorobenzyl)-4′-oxo-3′,4′-dihydro-1′H-spiro[piperidine-4,2′-quinoline]-1-carboxamide; and (b) at least one pharmaceutically acceptable carrier.

In one aspect, the present invention also provides a process for making crystalline Form A of 6′-fluoro-N-(4-fluorobenzyl)-4′-oxo-3′,4′-dihydro-1′H-spiro[piperidine-4,2′-quinoline]-1-carboxamide, said process comprising the steps of:
a) Reacting 6′-fluoro-1′H-spiro[piperidine-4,2′-quinolin]-4′(3′H)-one with 4-fluorobenzyl isocyanate in a chlorinated solvent, optionally in the presence of a base; and
b) Isolating the formed solid.

In a further aspect, the present invention also provides a process for making crystalline Form A of 6′-fluoro-N-(4-fluorobenzyl)-4′-oxo-3′,4′-dihydro-1′H-spiro[piperidine-4,2′-quinoline]-1-carboxamide, said process comprising the steps of:
a) Disolving an amount of 6′-fluoro-N-(4-fluorobenzyl)-4′-oxo-3′,4′-dihydro-1′H-spiro[piperidine-4,2′-quinoline]-1-carboxamide (especially of Form D) in a solvent;
b) Adding seed crystals of Form A of 6′-fluoro-N-(4-fluorobenzyl)-4′-oxo-3′,4′-dihydro-1′H-spiro[piperidine-4,2′-quinoline]-1-carboxamide; and
c) Isolating the formed solid.

In one aspect, the present invention also provides a method for the treatment or prevention of a disease or condition which may be treated by an AKR1C3 dependent KARS inhibitor, comprising administering to a patient in need of such treatment a therapeutically effective amount of crystalline Form A of 6′-fluoro-N-(4-fluorobenzyl)-4′-oxo-3′,4′-dihydro-1′H-spiro[piperidine-4,2′-quinoline]-1-carboxamide.

In one aspect, the present invention also provides the use of crystalline Form A of 6′-fluoro-N-(4-fluorobenzyl)-4′-oxo-3′,4′-dihydro-1′H-spiro[piperidine-4,2′-quinoline]-1-carboxamide for the preparation of a medicament.

In one aspect, the present invention also provides the use of crystalline Form A of 6′-fluoro-N-(4-fluorobenzyl)-4′-oxo-3′,4′-dihydro-1′H-spiro[piperidine-4,2′-quinoline]-1-carboxamide for the preparation of a medicament for the treatment of disorders which may be treated by an AKR1C3 dependent KARS inhibitor.

In one aspect, the present invention also provides crystalline Form A of 6′-fluoro-N-(4-fluorobenzyl)-4′-oxo-3′,4′-dihydro-1′H-spiro[piperidine-4,2′-quinoline]-1-carboxamide for use in the treatment of disorders which may be treated by an AKR1C3 dependent KARS inhibitor.

Accordingly, crystalline Form A of the compound of Formula (I) as described herein is useful in the treatment of cancer, in particular wherein the cancer is selected from solid tumors, non-small cell lung cancer (NSCLC), liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), and myelodysplastic syndrome (MDS).

The crystalline form of the compound of Formula (I) is especially useful in the treatment of non-small cell lung cancer (NSCLC).

Also described herein are further crystalline forms of 6′-fluoro-N-(4-fluorobenzyl)-4′-oxo-3′,4′-dihydro-1′H-spiro[piperidine-4,2′-quinoline]-1-carboxamide, designated as Forms B, C, D, E and hydrate form H_{A}. These crystalline forms may also be used, directly or indirectly, in the preparation of a medicament.

In a further aspect, the present invention also provides the use of a crystalline Form selected from A, B, C, D, E and hydrate form H_{A}, or mixtures thereof, of 6′-fluoro-N-(4-fluorobenzyl)-4′-oxo-3′,4′-dihydro-1′H-spiro[piperidine-4,2′-quinoline]-1-carboxamide for the preparation of a spray dried composition.

### BRIEF DESCRIPTION OF THE DRAWINGS

FIG. 1 provides an illustrative XRPD spectrum for an anhydrous crystalline form of compound of Formula (I), designated herein as Form A, showing degrees 2θ (2-theta) on the X-axis and relative intensity on the Y-axis.

FIG. 2 provides an illustrative DSC for an anhydrous crystalline form of the compound of Formula (I), designated herein as Form A.

FIG. 3 provides an illustrative TGA for an anhydrous crystalline form of the compound of Formula (I), designated herein as Form A.

FIG. 4 provides an illustrative XRPD spectrum for an anhydrous crystalline form of compound of Formula (I), designated herein as Form B, showing degrees 2θ (2-theta) on the X-axis and relative intensity on the Y-axis.

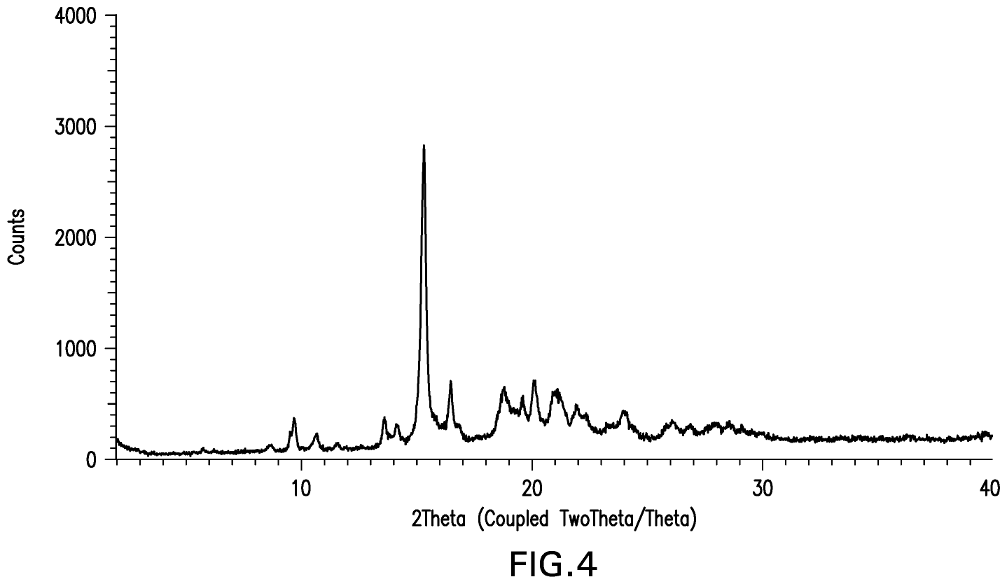

FIG. 5 provides an illustrative DSC for an anhydrous crystalline form of the compound of Formula (I), designated herein as Form B.

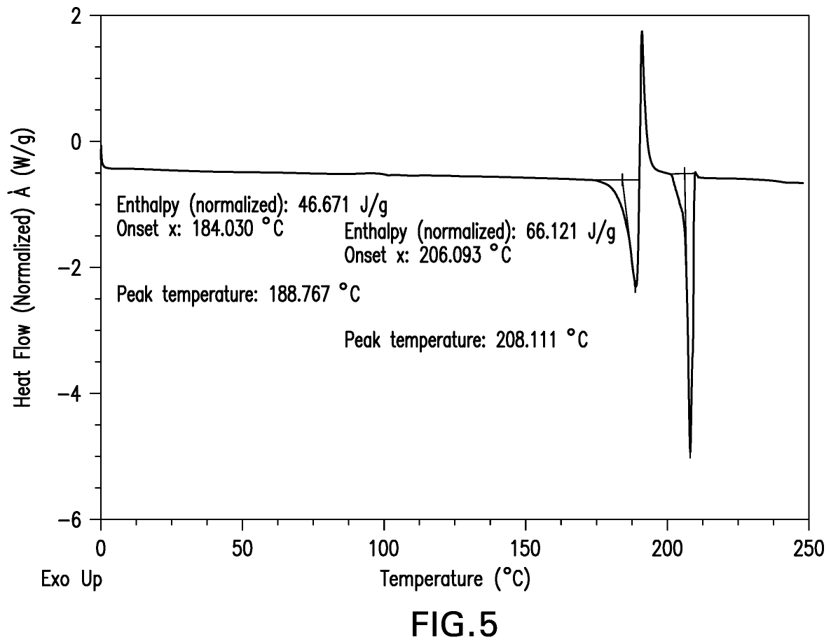

FIG. 6 provides an illustrative TGA for an anhydrous crystalline form of the compound of Formula (I), designated herein as Form B.

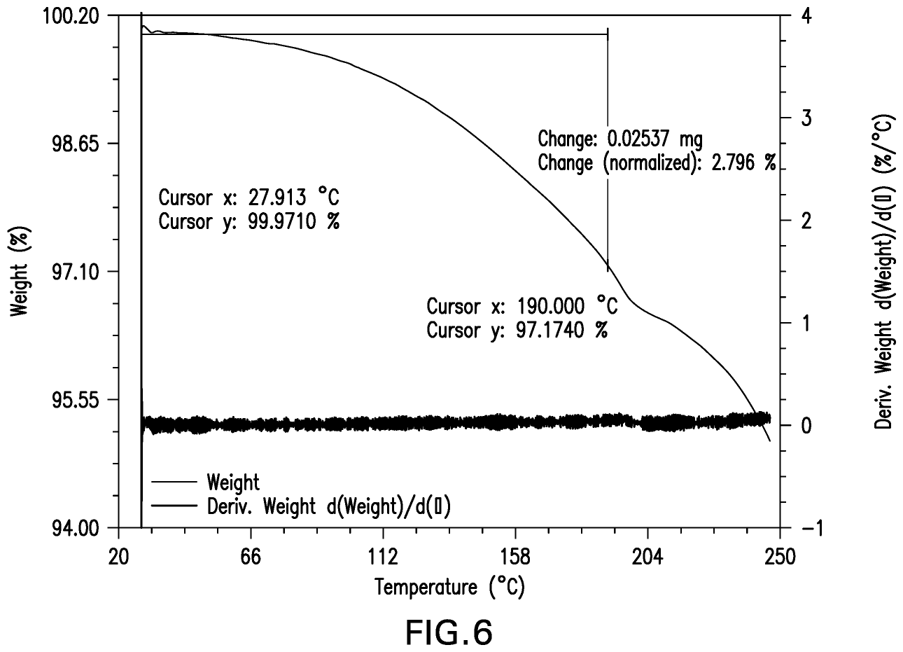

FIG. 7 provides an illustrative XRPD spectrum for an anhydrous crystalline form of compound of Formula (I), designated herein as Form C, showing degrees 2θ (2-theta) on the X-axis and relative intensity on the Y-axis.

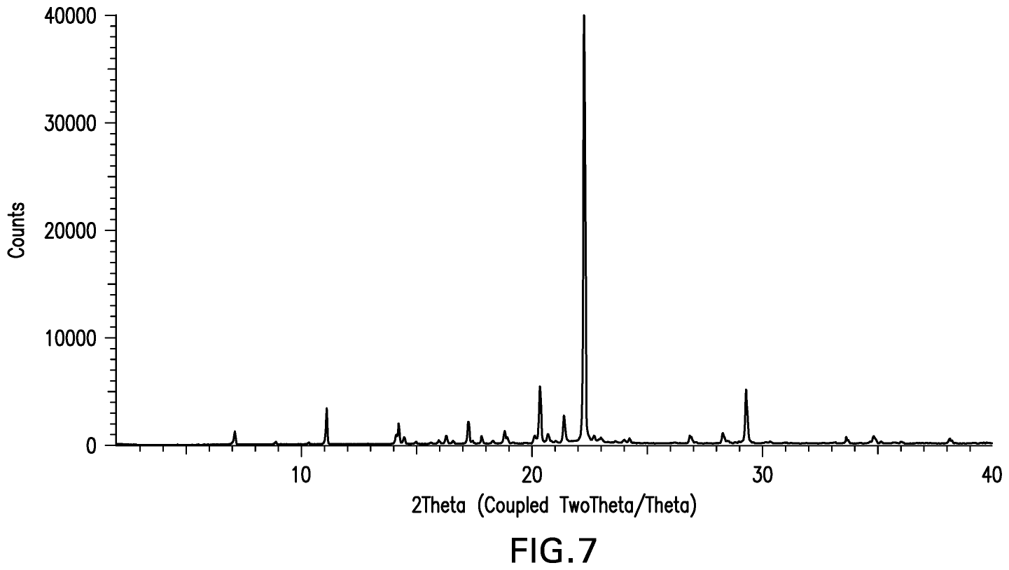

FIG. 8 provides an illustrative DSC for an anhydrous crystalline form of the compound of Formula (I), designated herein as Form C.

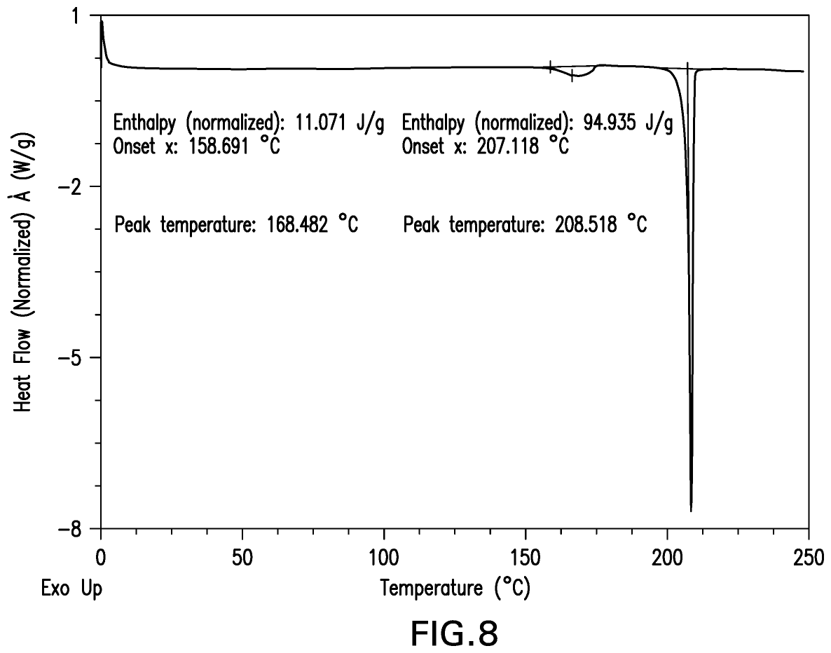

FIG. 9 provides an illustrative TGA for an anhydrous crystalline form of the compound of Formula (I), designated herein as Form C.

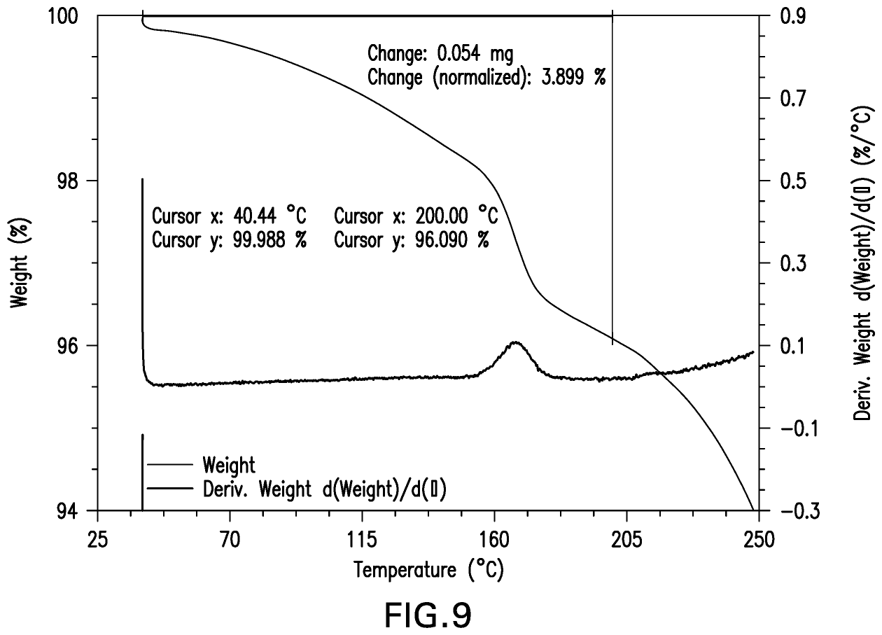

FIG. 10 provides an illustrative XRPD spectrum for an anhydrous crystalline form of compound of Formula (I), designated herein as Form D, showing degrees 2θ (2-theta) on the X-axis and relative intensity on the Y-axis.

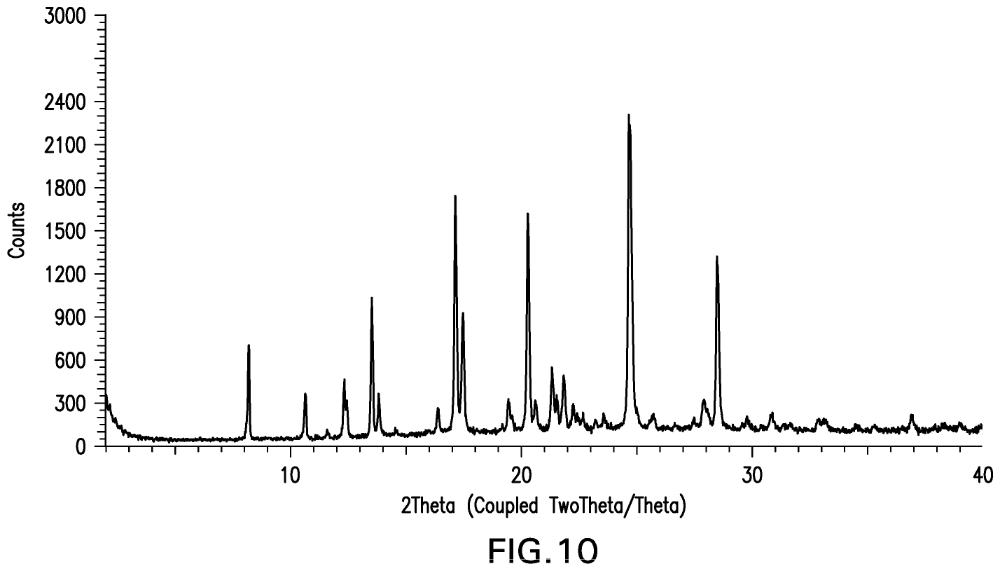

FIG. 11 provides an illustrative DSC for an anhydrous crystalline form of the compound of Formula (I), designated herein as Form D.

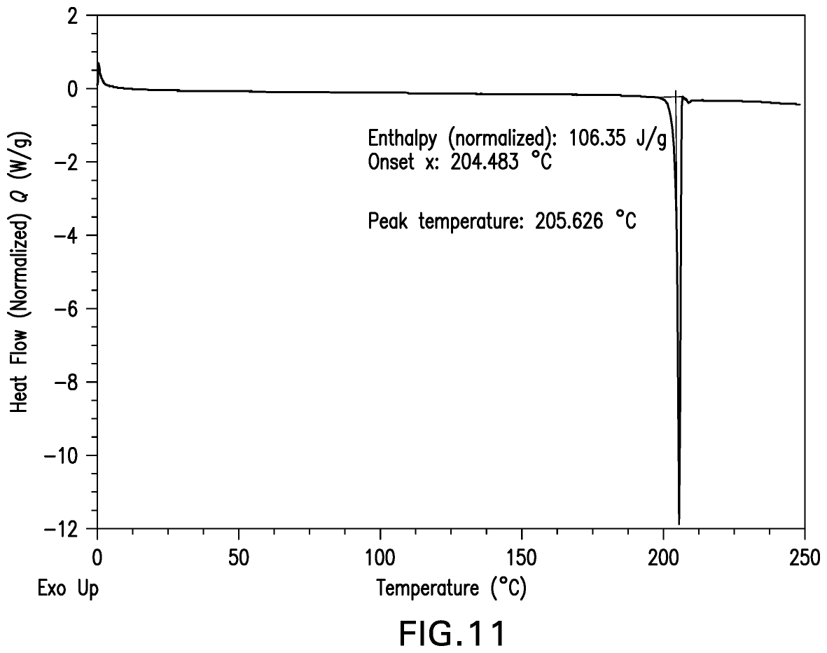

FIG. 12 provides an illustrative TGA for an anhydrous crystalline form of the compound of Formula (I), designated herein as Form D.

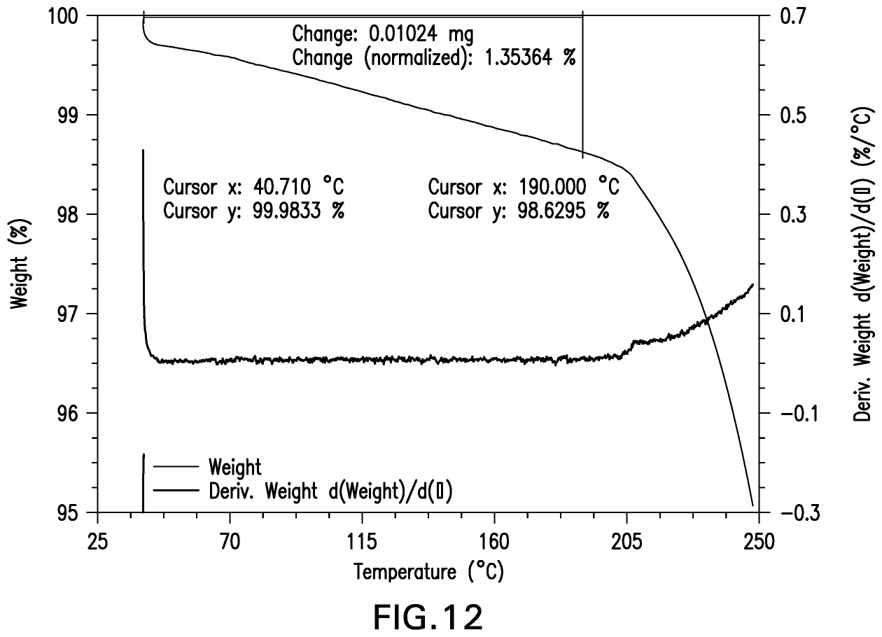

FIG. 13 provides an illustrative XRPD spectrum for an anhydrous crystalline form of compound of Formula (I), designated herein as Form E, showing degrees 2θ (2-theta) on the X-axis and relative intensity on the Y-axis.

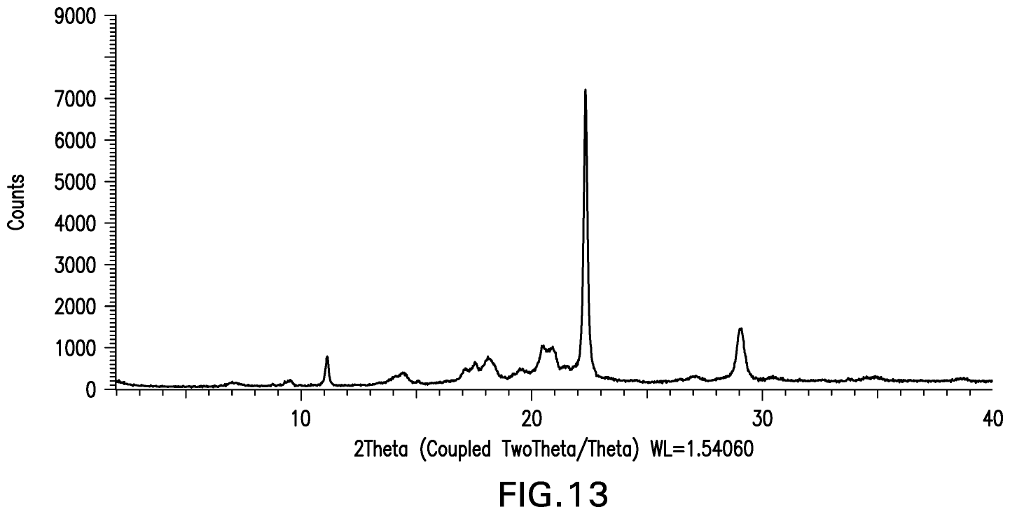

FIG. 14 provides an illustrative DSC for an anhydrous crystalline form of the compound of Formula (I), designated herein as Form E.

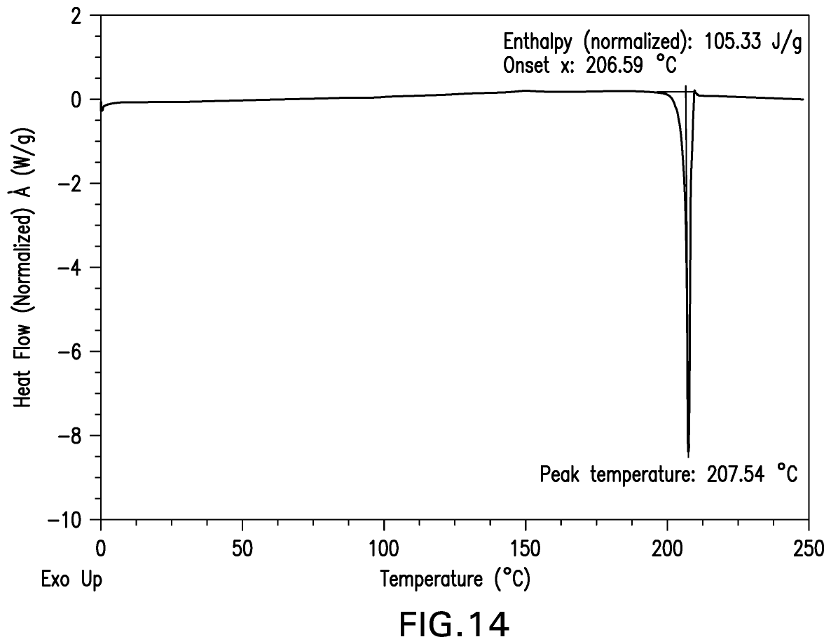

FIG. 15 provides an expanded portion of a DSC graph for an anhydrous crystalline form of the compound of Formula (I), designated herein as Form E.

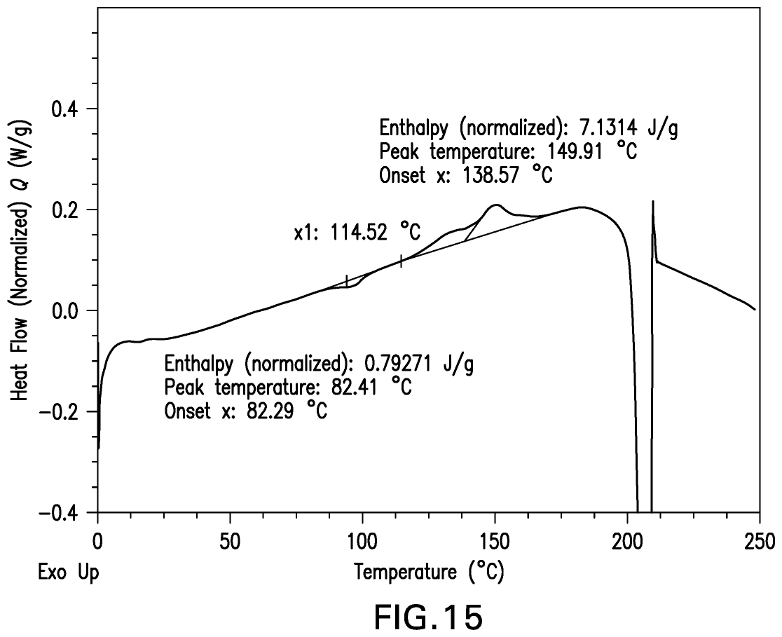

FIG. 16 provides an illustrative XRPD spectrum for an anhydrous crystalline form of compound of Formula (I), designated herein as hydrate Form H_{A}, showing degrees 2θ (2-theta) on the X-axis and relative intensity on the Y-axis.

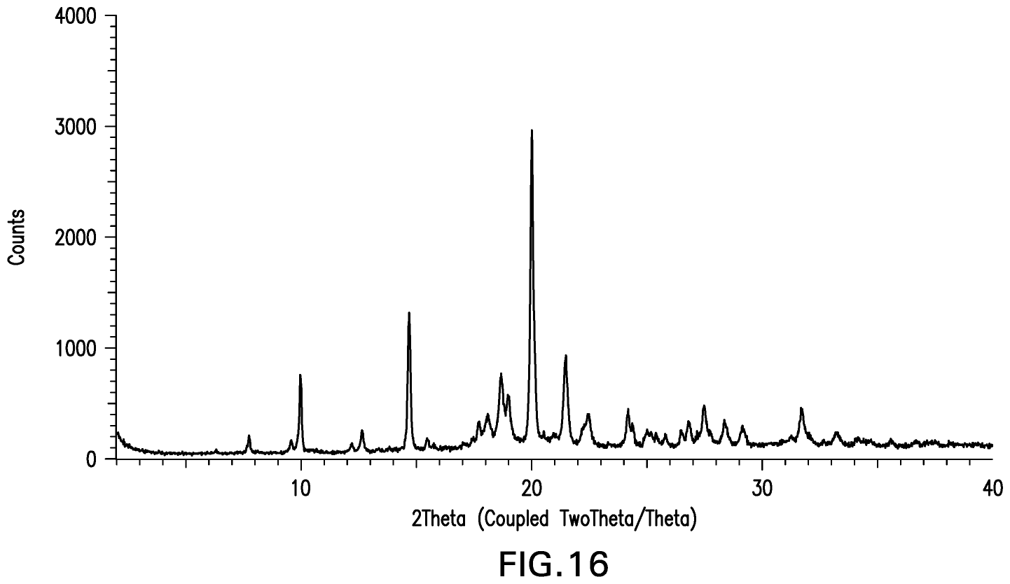

FIG. 17 provides an illustrative DSC for an anhydrous crystalline form of the compound of Formula (I), designated herein as hydrate Form H_{A}.

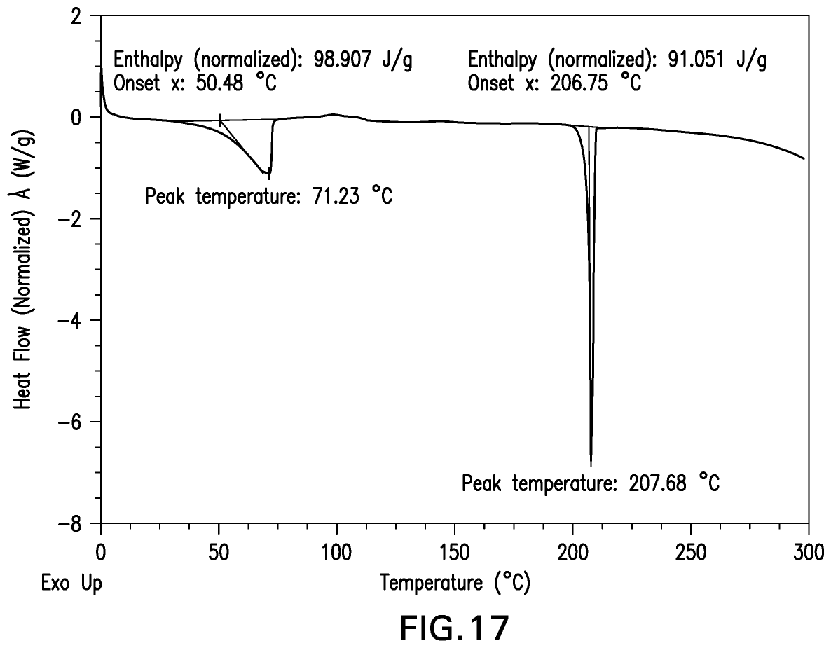

FIG. 18 provides an illustrative TGA for an anhydrous crystalline form of the compound of Formula (I), designated herein as hydrate Form H_{A}.

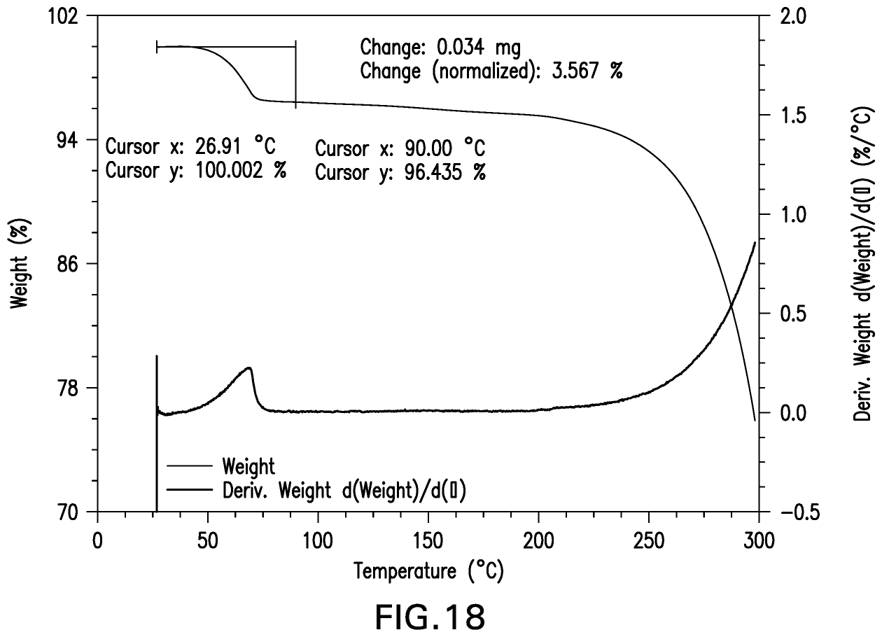

More detailed listings of the XRPD peaks for each of the forms are set forth in Tables 1 to 5 herein, in which the % relative intensity (I/I₀×100) is also provided. It should be understood that in the X-ray powder diffraction spectra or pattern that there is inherent variability in the values measured in degrees 2θ (2θ) as a result of, for example, instrumental variation (including differences between instruments). As such, it should be understood that there is a variability of up to ±0.2°2θ in XRPD peak measurements and yet such peak values would still be considered to be representative of a particular solid state form of the crystalline materials described herein. It should also be understood that other measured values from XRPD experiments and DSC/TGA experiments, such as relative intensity and water content, can vary as a result of, for example, sample preparation and/or storage and/or environmental conditions, and yet the measured values will still be considered to be representative of a particular solid state form of the crystalline materials described herein.

### DETAILED DESCRIPTION OF THE DISCLOSURE

### Definitions

As used herein, the terms “about” and “substantially” indicate with respect to features such as endotherms, endothermic peak, exotherms, baseline shifts, etc., that their values can vary. With reference to X-ray diffraction peak positions, “about” or “substantially” means that typical peak position and intensity variability are taken into account. For example, one skilled in the art will appreciate that the peak positions (2θ) will show some inter-apparatus variability, typically as much as 0.2°. Occasionally, the variability could be higher than 0.2° depending on apparatus calibration differences. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measure only. For DSC, variation in the temperatures observed will depend upon the rate of temperature change as well as sample preparation technique and the particular instrument employed. Thus, the endotherm/melting point values reported herein relating to DSC/TGA thermograms can vary ±5° C. (and still be considered to be characteristic of the particular crystalline form described herein). When used in the context of other features, such as, for example, percent by weight (% by weight), reaction temperatures, the term “about” indicates a variance of ±5%.

The terms “crystalline form(s)” or “crystalline modification(s)” or “polymorphic form(s)” or “polymorph(s)” will be used interchangeably herein. As used herein “polymorph” refers to crystalline forms having the same chemical composition but different spatial arrangements of the molecules, atoms, and/or ions forming the crystal. Each polymorph differs with respect to thermodynamic stability, physical parameters, x-ray structure and methods of preparation.

As used herein “amorphous” refers to a solid form of a molecule, atom, and/or ions that is not crystalline. An amorphous solid does not display a definitive X-ray diffraction pattern.

As used herein, “substantially pure,” when used in reference to a form, means a compound having a purity greater than 90 weight %, including greater than 90, 91, 92, 93, 94, 95, 96, 97, 98, and 99 weight %, and also including equal to about 100 weight % of compound of Formula (I), based on the weight of the compound. The remaining material comprises other form(s) of the compound, and/or reaction impurities and/or processing impurities arising from its preparation. For example, a crystalline form of the compound of Formula (I) may be deemed substantially pure in that it has a purity greater than 90 weight %, as measured by means that are at this time known and generally accepted in the art, where the remaining less than 10 weight % of material comprises other form(s) of the compound of Formula (I) and/or reaction impurities and/or processing impurities.

As used herein, “substantially phase pure,” when used in reference to any crystalline form of the compound of Formula (I), means a compound having a phase purity of greater than about 90% by weight, including greater than about 90, 91, 92, 93, 94, 95, 96, 97, 98, and about 99% by weight, and also including equal to about 100% by weight of the compound of Formula (I), based on the weight of the compound on an anhydrous basis. The term “phase pure” or “phase purity” herein refers to phase homogeneity with respect to a particular solid state form of the compound of Formula (I) and does not necessarily imply a high degree of chemical purity absent an express statement to that effect. Phase purity may be determined according to methods known in the art, for example, using XRPD to do quantitative phase analysis using one or more approaches known in the art, for example, via an external standard method, direct comparisons of line (peak) characteristics which are attributed to different phases in a particular spectra, or via an internal standard method. However XRPD quantification of phase purity can be complicated by the presence of amorphous material. Accordingly, other methods that may be useful for determining phase purity include, for example, solid state NMR spectroscopy, Raman and/or infrared spectroscopy. One of skilled in the art would readily understand these methods and how to employ these additional (or alternative) methods for determining phase purity.

As used herein, “substantially chemically pure” when used in reference to any crystalline form of the compound of Formula (I), means a compound having a chemical purity greater than about 90% by weight, including greater than about 90, 91, 92, 93, 94, 95, 96, 97, 98, and about 99% by weight, and also including equal to about 100% by weight of the compound of Formula (I), based on the weight of the compound on an anhydrous basis. The remaining material generally comprises other compounds, such as for example, other stereoisomers of the compound of Formula (I), reaction impurities, starting materials, reagents, side products, and/or other processing impurities arising from the preparation and/or isolation and/or purification of the particular crystalline form. For example, a crystalline form of the compound of Formula (I) may be deemed to be substantially chemically pure if it has been determined to have a chemical purity of greater than about 90% by weight, as measured by standard and generally accepted methods known in the art, where the remaining less than about 10% by weight constitutes other materials such as other stereoisomers of the compound of Formula (I), reaction impurities, starting materials, reagents, side products, and/or processing impurities. Chemical purity may be determined according to methods known in the art, for example, high performance liquid chromatography (HPLC), LC-MS (liquid chromatography-mass spectrometry), nuclear magnetic resonance (NMR) spectroscopy, or infrared spectroscopy. One of skill in the art would readily understand these methods and how to employ these additional (or alternative) methods for determining chemical purity.

As used herein, the term “seed” can be used as a noun to describe one or more crystals of a crystalline compound of Formula (I). The term “seed” can also be used as a verb to describe the act of introducing said one or more crystals of a crystalline compound of Formula (I) into an environment (including, but not limited to e.g., a solution, a mixture, a suspension, or a dispersion) thereby resulting in the formation of more crystals or the growth of the introduced crystals of the crystalline compound of Formula (I).

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by KARS, or (ii) disease sensitive to KARS inhibition, or (iii) characterized by activity (normal or abnormal) of KARS; or (2) reduce or inhibit disease sensitive to KARS inhibition. The invention further provides methods of treating, or preventing diseases and/or disorders related to high AKR1C3 expression or sensitivity to KARS inhibition, comprising administering to a subject in need thereof an effective amount of an AKR1C3 dependent KARS inhibitor.

As used herein, the term “subject” refers to primates (e.g., humans, male or female), monkeys, dogs, rabbits, guinea pigs, pigs, rats and mice. In certain embodiments, the subject is a primate. In yet other embodiments, the subject is a human.

As used herein, a subject is “in need of” or “in need thereof” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient. In one embodiment, “treat” or “treating” refers to delaying the progression of the disease or disorder.

As used herein, the term “prevent”, “preventing” or “prevention” of any disease or disorder refers to the prophylactic treatment of the disease or disorder; or delaying the onset of the disease or disorder.

The term “comprising” encompasses “including” as well as “consisting”; e.g., a composition comprising X may consist exclusively of X or may include an additional component, e.g. X and Y.

As used herein the term “combination” refers to either a fixed combination in one dosage unit form, or a combined administration where a crystalline form of compound of Formula (I) and a combination partner may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration.

The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “pharmaceutical combination” and “combination product” are used interchangeably and refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect. The term “fixed combination” means that a crystalline form of the compound of Formula (I) and a combination partner (i.e. immunotherapeutic agent), are both administered to a patient simultaneously in the form of a single entity or dosage. The term “non-fixed combination” means that a crystalline form of the compound of Formula (I) and a combination partner (i.e. the immunotherapeutic agent), are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent. In a preferred embodiment, the pharmaceutical combination is a non-fixed combination.

The term “combination therapy” refers to the administration of two or more therapeutic agents to treat a KARS related disease as described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

### Crystalline Forms:

The present disclosure relates to a crystalline form of 6′-fluoro-N-(4-fluorobenzyl)-4′-oxo-3′,4′-dihydro-1′H-spiro[piperidine-4,2′-quinoline]-1-carboxamide (the compound of Formula (I)), described and characterized herein.

In one embodiment, the present disclosure provides an anhydrous crystalline form of 6′-fluoro-N-(4-fluorobenzyl)-4′-oxo-3′,4′-dihydro-1′H-spiro[piperidine-4,2′-quinoline]-1-carboxamide.

In one embodiment, the present disclosure provides a crystalline form of 6′-fluoro-N-(4-fluorobenzyl)-4′-oxo-3′,4′-dihydro-1′H-spiro[piperidine-4,2′-quinoline]-1-carboxamide (Form A) having an X-ray powder diffraction (XRPD) pattern comprising a representative peak, in terms of °2θ, at 9.6±0.2°2θ measured at a temperature of about 25° C. In another embodiment, the XRPD pattern further comprises one or more additional representative peaks chosen from 17.1±0.2°2θ, 19.2±0.2°2θ and 21.0±0.2°2θ. In a further embodiment, the XRPD pattern further comprises one or more additional representative peaks chosen from 10.5±0.2°2θ and 30.4±0.2°2θ measured at a temperature of about 25° C. In a further embodiment, the XRPD pattern further comprises one or more additional representative peaks chosen from 13.4±0.2°2θ and 15.7±0.2°2θ measured at a temperature of about 25° C. In yet a further embodiment, the XRPD pattern further comprises one or more additional representative peaks chosen from 22.4±0.2°2θ, 27.3±0.2°2θ and 31.7±0.2°2θ measured at a temperature of about 25° C. Thus, the XRPD pattern for the crystalline Form A of the compound of Formula (I) may comprise one or more representative peaks selected from from the group consisting of 9.6±0.2°2θ, 10.5±0.2°2θ, 13.4±0.2°2θ, 15.7±0.2°2θ, 17.1±0.2°2θ, 19.2±0.2°2θ, 21.0±0.2°2θ, 22.4±0.2°2θ, 27.3±0.2°2θ, 30.4±0.2°2θ and 31.7±0.2°2θ measured at a temperature of about 25° C.

The XRPD pattern for the crystalline Form A may comprise one or more (e.g. two, three, four, five, six or seven) representative peaks selected from the peaks disclosed in table 1 and measured at a temperature of about 25° C.

In another aspect of the above embodiment, the crystalline Form A of compound of Formula (I) is characterized by a x-ray powder diffraction pattern comprising two or more 2θ values selected from the group consisting of 9.6±0.2°2θ, 10.5±0.2°2θ, 13.4±0.2°2θ, 15.7±0.2°2θ, 17.1±0.2°2θ, 19.2±0.2°2θ, 21.0±0.2°2θ, 22.4±0.2°2θ, 27.3±0.2°2θ, 30.4±0.2°2θ and 31.7±0.2°2θ, measured at a temperature of about 25° C. In another aspect of the above embodiment, the crystalline Form A of compound of Formula (I) is characterized by a x-ray powder diffraction pattern comprising three or more 2θ values (CuKα λ=1.54184 Å) selected from the group consisting of 9.6±0.2°2θ, 10.5±0.2°2θ, 13.4±0.2°2θ, 15.7±0.2°2θ, 17.1±0.2°2θ, 19.2±0.2°2θ, 21.0±0.2°2θ, 22.4±0.2°2θ, 27.3±0.2°2θ, 30.4±0.2°2θ and 31.7±0.2°2θ measured at a temperature of about 25° C. In another aspect of the above embodiment, the crystalline Form A of compound of Formula (I) is characterized by a x-ray powder diffraction pattern comprising four or more 2θ values selected from the group consisting of 9.6±0.2°2θ, 10.5±0.2°2θ, 13.4±0.2°2θ, 15.7±0.2°2θ, 17.1±0.2°2θ, 19.2±0.2°2θ, 21.0±0.2°2θ, 22.4±0.2°2θ, 27.3±0.2°2θ, 30.4±0.2°2θ and 31.7±0.2°2θ measured at a temperature of about 25° C. In another aspect of the above embodiment, the crystalline Form A of compound of Formula (I) is characterized by a x-ray powder diffraction pattern comprising five or more 2θ values selected from the group consisting of 9.6±0.2°2θ, 10.5±0.2°2θ, 13.4±0.2°2θ, 15.7±0.2°2θ, 17.1±0.2°2θ, 19.2±0.2°2θ, 21.0±0.2°2θ, 22.4±0.2°2θ, 27.3±0.2°2θ, 30.4±0.2°2θ and 31.7±0.2°2θ measured at a temperature of about 25° C.

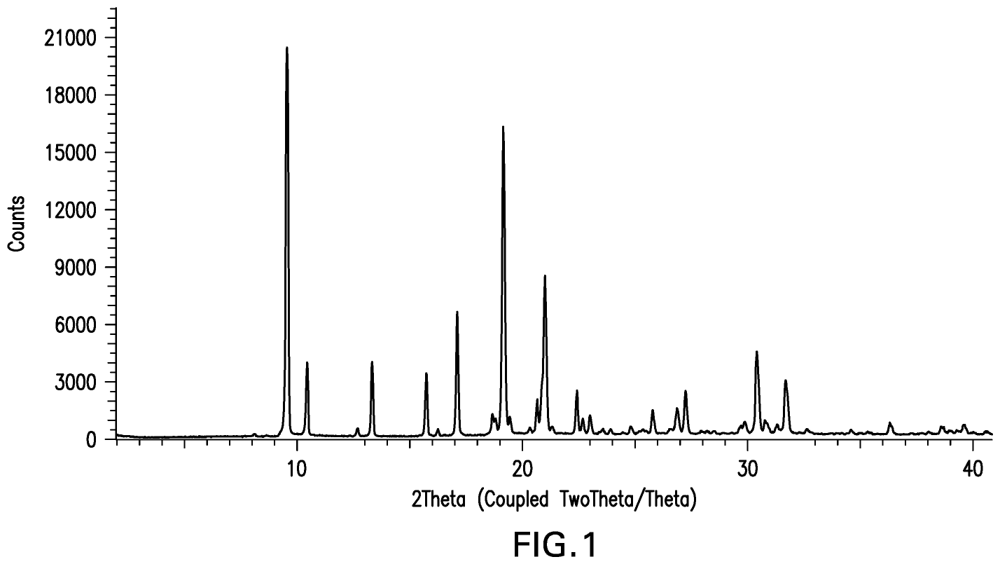

In yet another aspect of the above embodiment, the crystalline Form A of the compound of Formula (I) has an XRPD pattern substantially as shown in FIG. 1.

The crystalline Form A of 6′-fluoro-N-(4-fluorobenzyl)-4′-oxo-3′,4′-dihydro-1′H-spiro[piperidine-4,2′-quinoline]-1-carboxamide may be characterized thermally. In one embodiment, crystalline Form A of the compound of Formula (I) has a thermal profile measured by Differential Scanning calorimetry (DSC) with a heating rate of 10° C./min comprising a single endothermic peak starting at about 208° C. (corresponding to melting).

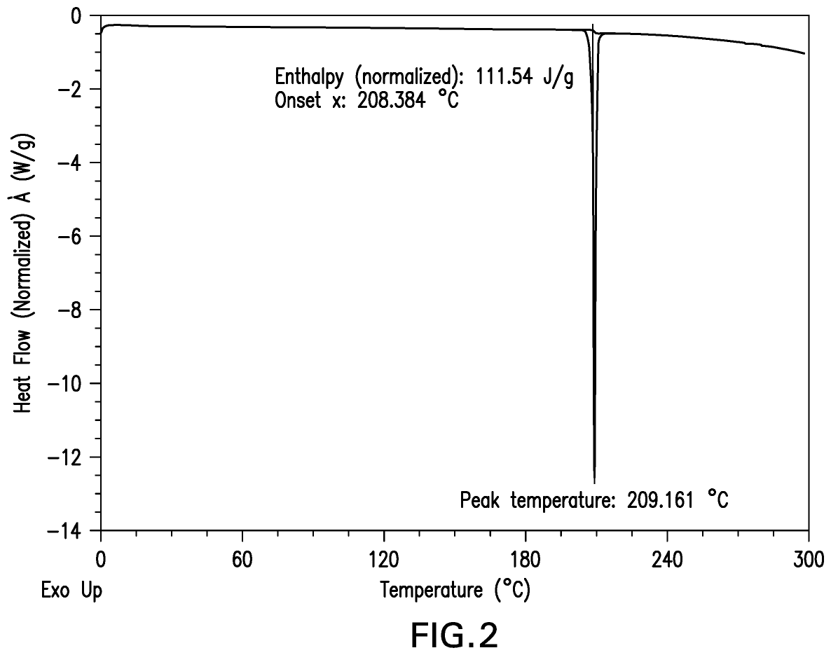

In another embodiment, the crystalline Form A of the compound of Formula (I) has a DSC thermogram that is substantially as shown in FIG. 2. It should be understood that hydrated forms may yield different thermograms (in terms of peak shape and profile) depending on instrument parameters, thus the same material may have thermograms that look substantially different from each other when the data is generated on two different instruments.

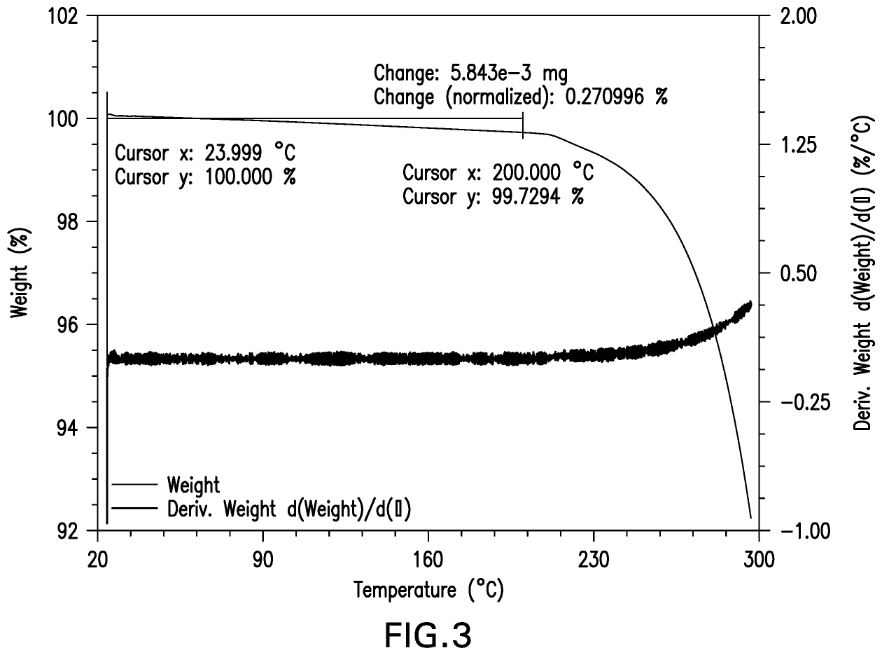

In another embodiment, the crystalline Form A of the compound of Formula (I) has a thermogravimetric analysis (TGA) diagram substantially the same as that shown in FIG. 3. The weight loss by TGA is about 0.3% in the range of about 24-200° C. Thermal decomposition occurred at 240° C.

In yet another embodiment, the crystalline Form A is substantially pure.

In yet another embodiment, the crystalline Form A is substantially chemically pure.

In yet another embodiment, the crystalline Form A is substantially phase pure.

In one aspect, the present invention also provides a process for making crystalline Form A of 6′-fluoro-N-(4-fluorobenzyl)-4′-oxo-3′,4′-dihydro-1′H-spiro[piperidine-4,2′-quinoline]-1-carboxamide, said process comprising the steps of:
a) Reacting 6′-fluoro-1′H-spiro[piperidine-4,2′-quinolin]-4′(3′H)-one with 4-fluorobenzyl isocyanate in a chlorinated solvent, optionally in the presence of a base; and
b) Isolating the formed solid.

In an embodiment of said process, the reaction is performed in the presence of a base. In an embodiment, said base is an amine. In another embodiment, said base is a tertiary amine. In another embodiment, said base is N, N-Diisopropylethylamine.

In an embodiment of said process, the chlorinated solvent is dichloromethane.

In an embodiment of said process, the reaction is carried out at a temperature of about 20° C. to about 50° C. In an embodiment of said process, the reaction is carried out at a temperature of about 20° C. to about 30° C. In an embodiment of said process, the reaction is carried out at a temperature of about 23° C. to about 28° C.

In a further aspect, the present invention also provides a process for making crystalline Form A of 6′-fluoro-N-(4-fluorobenzyl)-4′-oxo-3′,4′-dihydro-1′H-spiro[piperidine-4,2′-quinoline]-1-carboxamide, said process comprising the steps of:

a) Dissolving an amount of 6′-fluoro-N-(4-fluorobenzyl)-4′-oxo-3′,4′-dihydro-1′H-spiro[piperidine-4,2′-quinoline]-1-carboxamide (especially of Form D) in a solvent;

b) Adding seed crystals of Form A of 6′-fluoro-N-(4-fluorobenzyl)-4′-oxo-3′,4′-dihydro-1′H-spiro[piperidine-4,2′-quinoline]-1-carboxamide; and

c) Isolating the formed solid.

In an embodiment of said further process the solvent comprises a non-chlorinated solvent.

In an embodiment of said process said non-chlorinated solvent is selected from THF, acetone, water, acetonitrile, dioxane, ethanol, methanol, butanone or mixtures thereof. In another embodiment of said process, said solvent comprises a mixture of solvents comprising a non-chorinated solvent. In an embodiment said mixture of solvents comprises acetone and water. In an embodiment the solvent in step a) of said process is a mixture of acetone:water 85:15 w/w. In an embodiment, step a) of said process is carried out at a temperature of about 50° C. to about 60° C. (preferably about 50° C.).

In an embodiment of said process, the seed crystals in step b) are added as a dispersation in a mixture of acetone and water. In an embodiment of said process, said dispersation is in a mixture of 40:60 w/w acetone:water. In an embodiment of said process, step b) is carried out at a temperature of about 40° C., optionally followed by cooling to 20° C., optionally followed by addition of water to bring the solvent mixture to 40:60 w/w acetone:water.

In another aspect, the invention relates to a pharmaceutical composition comprising a therapeutically effective amount of a crystalline form of 6′-fluoro-N-(4-fluorobenzyl)-4′-oxo-3′,4′-dihydro-1′H-spiro[piperidine-4,2′-quinoline]-1-carboxamide and at least one pharmaceutically acceptable carrier, diluent or excipient. In a particular embodiment of this aspect, the invention relates to a pharmaceutical composition comprising crystalline Form A, and one or more pharmaceutically acceptable carriers, diluents or excipients. In another embodiment, the invention relates to a pharmaceutical composition comprising crystalline Form A in substantially phase pure form.

In yet another embodiment, the invention relates to a pharmaceutical composition comprising crystalline Form A and further comprising at least one other solid state form of 6′-fluoro-N-(4-fluorobenzyl)-4′-oxo-3′,4′-dihydro-1′H-spiro[piperidine-4,2′-quinoline]-1-carboxamide.

In another aspect, the invention relates to combinations, in particular pharmaceutical combinations, comprising a therapeutically effective amount of a crystalline form of 6′-fluoro-N-(4-fluorobenzyl)-4′-oxo-3′,4′-dihydro-1′H-spiro[piperidine-4,2′-quinoline]-1-carboxamide, and one or more other therapeutic agents. In a particular embodiment of this aspect, the invention relates to a pharmaceutical combination comprising crystalline Form A, and one or more other therapeutic agents. In another embodiment, the invention relates to a pharmaceutical combination comprising crystalline Form A in substantially phase pure form and one or more other therapeutic agents. In another embodiment, the invention relates to a pharmaceutical combination comprising crystalline Form A in substantially phase pure form and one or more other therapeutic agents.

In yet another embodiment, the invention relates to a pharmaceutical combination comprising crystalline Form A and further comprising at least one other solid state form of 6′-fluoro-N-(4-fluorobenzyl)-4′-oxo-3′,4′-dihydro-1′H-spiro[piperidine-4,2′-quinoline]-1-carboxamide in addition to one or more other therapeutic agents.

In another embodiment, the invention provides pharmaceutical combinations as described herein wherein the other therapeutic agent is independently selected from the group of anti-cancer or chemotherapeutic agents, anti-nausea agents (or anti-emetics), a chemotherapy, pain relievers, cytoprotective agents, and combinations thereof.

In some embodiments, the compounds of Formula (I), or a pharmaceutically acceptable salt thereof, of the present disclosure are administered in combination with one or more second agent(s) selected from a PD-1 inhibitor, a PD-L1 inhibitor, a LAG-3 inhibitor, a cytokine, an A2A antagonist, a GITR agonist, a TIM-3 inhibitor, a STING agonist, and a TLR7 agonist, to treat a disease, e.g., cancer.

In another embodiment, one or more chemotherapeutic agents are used in combination with the compounds of Formula (I), or a pharmaceutically acceptable salt, thereof, for treating a disease, e.g., cancer, wherein said chemotherapeutic agents include, but are not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-UR), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide f (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), epirubicin (Ellence®), oxaliplatin (Eloxatin®), exemestane (Aromasin®), letrozole (Femara®), and fulvestrant (Faslodex®).

In other embodiments, the compounds of Formula (I), or a pharmaceutically acceptable salt, thereof, of the present disclosure are used in combination with one or more other anti-HER2 antibodies, e.g., trastuzumab, pertuzumab, margetuximab, or HT-19 described above, or with other anti-HER2 conjugates, e.g., ado-trastuzumab emtansine (also known as Kadcyla®, or T-DM1).

In other embodiments, the compounds of Formula (I), or a pharmaceutically acceptable salt, thereof, of the present disclosure are used in combination with one or more tyrosine kinase inhibitors, including but not limited to, EGFR inhibitors, Her3 inhibitors, IGFR inhibitors, and Met inhibitors, for treating a disease, e.g., cancer.

For example, tyrosine kinase inhibitors include but are not limited to, Erlotinib hydrochloride (Tarceva®); Linifanib (N-[4-(3-amino-1H-indazol-4-yl)phenyl]-N′-(2-fluoro-5-methylphenyl) urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl) propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (Gilotrif®); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS187724-61-4).

EGFR antibodies include but are not limited to, Cetuximab (Erbitux®); Panitumumab (Vectibix®); Matuzumab (EMD-72000); Nimotuzumab (hR3); Zalutumumab; TheraCIM h-R3; MDX0447 (CAS 339151-96-1); and ch806 (mAb-806, CAS 946414-09-1).

Other HER2 inhibitors include but are not limited to, Neratinib (HKI-272, (2E)-N-[4-[[3-chloro-4-[(pyridin-2-yl) methoxy]phenyl]amino]-3-cyano-7-ethoxyquinolin-6-yl]-4-(dimethylamino) but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8).

HER3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl) carbonyl]-1H-pyrrol-2-yl}methylene)-1,3-dihydro-2H-indol-2-one (SU11271); (3Z)—N-(3-Chlorophenyl)-3-({3,5-dimethyl-4-[(4-methylpiperazin-1-yl) carbonyl]-1H-pyrrol-2-yl}methylene)-N-methyl-2-oxoindoline-5-sulfonamide (SU11274); (3Z)—N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1Hpyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin 3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

IGFR inhibitors include but are not limited to, BMS-754807, XL-228, OSI-906, GSK0904529A, A-928605, AXL1717, KW-2450, MK0646, AMG479, IMCA12, MEDI-573, and BI836845. See e.g., Yee, JNCI, 104; 975 (2012) for review.

In another embodiment, the compounds of Formula (I) of the present disclosure are used in combination with one or more proliferation signalling pathway inhibitors, including but not limited to, MEK inhibitors, BRAF inhibitors, PI3K/Akt inhibitors, SHP2 inhibitors, and also mTOR inhibitors, and CDK inhibitors, for treating a disease, e.g., cancer.

For example, mitogen-activated protein kinase (MEK) inhibitors include but are not limited to, XL-518 (also known as GDC-0973, CAS No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

BRAF inhibitors include, but are not limited to, Vemurafenib (or Zelboraf®), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®), or Ipilimumab (or MDX-010, MDX-101, or Yervoy).

Phosphoinositide 3-kinase (PI3K) inhibitors include, but are not limited to, 4-[2-(1H-Indazol-4-yl)-6-[[4-(methylsulfonyl) piperazin-1-yl]methyl]thieno[3,2-d]pyrimidin-4-yl]morpholine (also known as GDC0941, RG7321, GNE0941, Pictrelisib, or Pictilisib; and described in PCT Publication Nos. WO 09/036082 and WO 09/055730); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethylcyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); (S)—N1-(4-methyl-5-(2-(1,1,1-trifluoro-2-methylpropan-2-yl) pyridin-4-yl) thiazol-2-yl) pyrrolidine-1,2-dicarboxamide (also known as BYL719 or Alpelisib); 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide (also known as GDC0032, RG7604, or Taselisib).

mTOR inhibitors include but are not limited to, Temsirolimus (Torisel®); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl) methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl) morpholinium-4-yl]methoxy]butyl]-L-arginylglycyl-L-□-aspartyIL-serine-, inner salt (SF1126, CAS 936487-67-1).

CDK inhibitors include but are not limited to, Palbociclib (also known as PD-0332991, Ibrance®, 6-Acetyl-8-cyclopentyl-5-methyl-2-{[5-(1-piperazinyl)-2-pyridinyl]amino}pyrido[2,3-d]pyrimidin-7(8H)-one).

In yet another embodiment, the compounds of Formula (I), or a pharmaceutically acceptable salt, hydrate, solvate, prodrug, stereoisomer, or tautomer thereof, of the present disclosure are used in combination with one or more pro-apoptotics, including but not limited to, IAP inhibitors, BCL2 inhibitors, MCL1 inhibitors, TRAIL agents, CHK inhibitors, for treating a disease, e.g., cancer.

For examples, IAP inhibitors include but are not limited to, LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, US2006/0014700, US2006/0025347, WO 06/069063, WO 06/010118, WO 06/017295, and WO08/134679, all of which are incorporated herein by reference.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−) BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab (CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinylpyrazolo[1,5-a]pyrimidin-7-amine (SCH900776, CAS 891494-63-6); 5-(3-Fluorophenyl)-3-ureidothiophene-2-carboxylic acid N—[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2 (1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl) urea (LY2603618, CAS 911222-45-2); Sulforaphane (CAS 4478-93-7, 4-Methylsulfinylbutyl isothiocyanate); 9,10,11,12-Tetrahydro-9,12-epoxy-1H-diindolo[1,2,3-fg:3′,2′,1′-kl]pyrrolo[3,4-t][1,6]benzodiazocine-1,3(2H)-dione (SB-218078, CAS 135897-06-2); and TAT-S216A (YGRKKRRQRRRLYRSPAMPENL (SEQ ID NO: 33)), and CBP501 ((d-Bpa)sws(d-Phe-F5)(d-Cha)rrrqrr).

In one embodiment, the invention relates to a method for the treatment or prevention of a disease or condition which may be treated by an AKR1C3 dependent KARS inhibitor (e.g. cancer), in a subject in need thereof, the method comprising: administering to a subject in need thereof, a therapeutically effective amount of a crystalline form of 6′-fluoro-N-(4-fluorobenzyl)-4′-oxo-3′,4′-dihydro-1′H-spiro[piperidine-4,2′-quinoline]-1-carboxamide (preferably Form A), alone or in combination with one or more other therapeutic agents.

In another embodiment, the invention relates to a method for the treatment or prevention of a disease or condition which may be treated by an AKR1C3 dependent KARS inhibitor (e.g. cancer), in a subject in need thereof, the method comprising: administering to said subject, a pharmaceutical composition as described herein, alone or in combination with one or more other therapeutic agents.

In another embodiment, the invention relates to a method for the treatment or prevention of a disease or condition which may be treated by an AKR1C3 dependent KARS inhibitor (e.g. cancer), in a subject in need thereof, comprising administering to said subject a pharmaceutical combination as described herein.

In one embodiment, the invention relates to the use of a crystalline form of 6′-fluoro-N-(4-fluorobenzyl)-4′-oxo-3′,4′-dihydro-1′H-spiro[piperidine-4,2′-quinoline]-1-carboxamide (preferably Form A), alone or in combination with one or more other therapeutic agents, for the treatment or prevention of a disease or condition which may be treated by an AKR1C3 dependent KARS inhibitor (e.g. cancer).

In yet another embodiment, the invention pertains to a crystalline form of 6′-fluoro-N-(4-fluorobenzyl)-4′-oxo-3′,4′-dihydro-1′H-spiro[piperidine-4,2′-quinoline]-1-carboxamide (preferably Form A), for use in the treatment or prevention of a disease or condition which may be treated by an AKR1C3 dependent KARS inhibitor (e.g. cancer).

In yet another embodiment, the invention pertains to a combination of a crystalline form of 6′-fluoro-N-(4-fluorobenzyl)-4′-oxo-3′,4′-dihydro-1′H-spiro[piperidine-4,2′-quinoline]-1-carboxamide (preferably Form A), and one or more other therapeutic agents, for use in the treatment or prevention of a disease or condition which may be treated by an AKR1C3 dependent KARS inhibitor (e.g. cancer).

In one embodiment, the invention relates to a method of treatment, a use, a compound for use, or a combination for use as described herein, wherein the disease or disorder which may be treated by an AKR1C3 dependent KARS inhibitor, is selected from non-small cell lung cancer (NSCLC), liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), and myelodysplastic syndrome (MDS).

The characterisation of Form A is described herein and with reference to the figures.

Further crystalline forms of 6′-fluoro-N-(4-fluorobenzyl)-4′-oxo-3′,4′-dihydro-1′H-spiro[piperidine-4,2′-quinoline]-1-carboxamide have been observed, designated herein as Form B, C, D and E. A hydrate form, designated herein as H_{A} was also observed. The characterisation of these further forms is described herein and with reference to the figures.

In one aspect, the present invention also provides the use of a crystalline Form selected from A, B, C, D, E and hydrate form H_{A}, or mixtures thereof, of 6′-fluoro-N-(4-fluorobenzyl)-4′-oxo-3′,4′-dihydro-1′H-spiro[piperidine-4,2′-quinoline]-1-carboxamide for the preparation of a medicament.

In one aspect, the present invention also provides the use of a crystalline Form selected from A, B, C, D, E and hydrate form H_{A}, or mixtures thereof, of 6′-fluoro-N-(4-fluorobenzyl)-4′-oxo-3′,4′-dihydro-1′H-spiro[piperidine-4,2′-quinoline]-1-carboxamide for the preparation of a spray dried composition. In an embodiment of this aspect, the invention provides the use of crystalline Form A for the preparation of a spray dried composition. Said composition can be used in the preparation of a medicament. Said medicament may comprise said spray dried composition.

Said spray dried composition may be prepared by dissolving a crystalline Form selected from A, B, C, D, E and hydrate form H_{A}, or mixtures thereof, in a suitable solvent and subjecting the solution to spray drying. In one embodiment, a suitable solvent is an organic solvent, water, or a combination thereof. In another embodiment, a suitable organic solvent includes, but is not limited to acetone, ethanol, methanol, and propanol. In a further embodiment, a suitable solvent comprises a mixture of acetone and water.

### Pharmaceutical Composition, Dosage and Administration

In one embodiment the crystalline forms of 6′-fluoro-N-(4-fluorobenzyl)-4′-oxo-3′,4′-dihydro-1′H-spiro[piperidine-4,2′-quinoline]-1-carboxamide described herein can be used alone or they can be formulated into a pharmaceutical composition that also contains at least one pharmaceutically acceptable excipient, and often contains at least two or more pharmaceutically acceptable excipients. Some suitable excipients are disclosed herein. Other excipients may be used that are known in the art without departing from the intent and scope of the present application.

In some embodiments, the present invention utilizes a pharmaceutical composition comprising a compound of the present invention and a pharmaceutically acceptable excipient.

As used herein, the term “pharmaceutically acceptable excipients” includes any and all solvents, carriers, diluents, dispersion media, coatings, surfactants, antioxidants, preservatives (e.g., antibacterial agents, antifungal agents, antioxidants), isotonic agents, absorption delaying agents, salts, drug stabilizers, binders, additives, bulking agents, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). It should be understood that unless a conventional excipient is incompatible with the active ingredient, the use of any conventional excipient in any therapeutic or pharmaceutical compositions is contemplated by the present application.

The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, carriers or buffering agents, as well as adjuvants, such as solvents, preservatives, stabilizers, wetting agents, emulsifiers and bulking agents, etc.

Typically, the pharmaceutical compositions are tablets or capsules comprising the active ingredient together with at least one excipient, such as:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired;
d) carriers such as an aqueous vehicle containing a co-solvating material such as captisol, PEG, glycerin, cyclodextrin, or the like;
e) disintegrants, e.g., starches, agar, alginic acid or its sodium salt, or effervescent mixtures; and/or
f) absorbents, colorants, flavors and sweeteners.

Tablets may be either film coated or enteric coated according to methods known in the art.

Preferably, the compound or composition is prepared for oral administration, such as a tablet or capsule, for example, and optionally packaged in a multi-dose format suitable for storing and/or dispensing unit doses of a pharmaceutical product. Examples of suitable packaging include, but are not limited to, hermetically sealed foils, unit dose containers (e.g., vials), blister packs, and strip packs.

Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 10-50 mg of active ingredients. Preferably, the pharmaceutical composition or combination of the present invention can be in unit dosage of about 10 mg, about 25 mg or about 50 mg. The therapeutically effective dosage or amount of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg. Preferably, the therapeutically effective amount in vivo ranges between about 10 mg to about 200 mg daily, for example, about 10 mg, about 20 mg, about 25 mg, about 35 mg, about 50 mg, about 100 mg or about 200 mg daily. Preferably, the therapeutically effective amount in vivo is selected from about 10 mg, about 35 mg, about 50 mg or about 100 mg once a day. Also, preferably, the therapeutically effective amount in vivo is selected from about 10 mg, about 25 mg, about 50 mg or about 100 mg twice a day.

In other embodiments, a pharmaceutical composition is provided which comprises at least one crystalline form according to the embodiments herein supra (e.g. Form A, Form E, Form H_{A}, or mixtures thereof, preferably Form A); and at least one pharmaceutically acceptable carrier.

Accordingly, in an embodiment of the disclosure, a crystalline form of 6′-fluoro-N-(4-fluorobenzyl)-4′-oxo-3′,4′-dihydro-1′H-spiro[piperidine-4,2′-quinoline]-1-carboxamide (Form A, Form E or Form H_{A}, or preferably Form A) is provided in a substantially phase pure form. This crystalline form of 6′-fluoro-N-(4-fluorobenzyl)-4′-oxo-3′,4′-dihydro-1′H-spiro[piperidine-4,2′-quinoline]-1-carboxamide (Form A, Form E or Form H_{A}, or preferably Form A) in substantially phase pure form may be used to prepare pharmaceutical compositions which may further comprise one or more pharmaceutically acceptable excipients.

### Combination:

The crystalline form of 6′-fluoro-N-(4-fluorobenzyl)-4′-oxo-3′,4′-dihydro-1′H-spiro[piperidine-4,2′-quinoline]-1-carboxamide (preferably Form A) may be administered either simultaneously with, or before or after, one or more other therapeutic agents. The crystalline form of the present invention may be administered separately, by the same or different route of administration, or together in the same pharmaceutical composition as the other agents.

The crystalline form of 6′-fluoro-N-(4-fluorobenzyl)-4′-oxo-3′,4′-dihydro-1′H-spiro[piperidine-4,2′-quinoline]-1-carboxamide (preferably Form A) may be administered as the sole active ingredient or in conjunction with, e.g. as an adjuvant to, other drugs e.g. anti-cancer or chemotherapeutic agents, anti-nausea agents (or anti-emetics), a chemotherapy, pain relievers, cytoprotective agents, and combinations thereof. For example, the crystalline form of 6′-fluoro-N-(4-fluorobenzyl)-4′-oxo-3′,4′-dihydro-1′H-spiro[piperidine-4,2′-quinoline]-1-carboxamide may be used in combination with anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BICNUR), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

### Therapeutic Kits

In one embodiment, the invention provides a kit comprising two or more separate pharmaceutical compositions, at least one of which contains a crystalline form of 6′-fluoro-N-(4-fluorobenzyl)-4′-oxo-3′,4′-dihydro-1′H-spiro[piperidine-4,2′-quinoline]-1-carboxamide (preferably Form A). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, a crystalline form of 6′-fluoro-N-(4-fluorobenzyl)-4′-oxo-3′,4′-dihydro-1′H-spiro[piperidine-4,2′-quinoline]-1-carboxamide (preferably Form A) and the other therapeutic agent may be manufactured and/or formulated by the same or different manufacturers. Moreover, a crystalline form of 6′-fluoro-N-(4-fluorobenzyl)-4′-oxo-3′,4′-dihydro-1′H-spiro[piperidine-4,2′-quinoline]-1-carboxamide (preferably Form A) and the other therapeutic may be brought together into a combination therapy: (i) prior to release of the combination product to physicians (e.g. in the case of a kit comprising a crystalline form of compound of Formula (I) and the other therapeutic agent); (ii) by the physician themselves (or under the guidance of the physician) shortly before administration; (iii) in the patient themselves, e.g. during sequential administration of a crystalline form of the compound of Formula (I) and the other therapeutic agent.

Accordingly, the invention provides the use of a crystalline form of 6′-fluoro-N-(4-fluorobenzyl)-4′-oxo-3′,4′-dihydro-1′H-spiro[piperidine-4,2′-quinoline]-1-carboxamide (preferably Form A), for treating a disease or condition which may be treated by an AKR1C3 dependent KARS inhibitor (e.g. cancer), wherein the medicament is prepared for administration with another therapeutic agent. The invention also provides the use of a therapeutic agent for treating a disease or condition which may be treated by an AKR1C3 dependent KARS inhibitor (e.g. cancer), wherein the medicament is administered with a crystalline form of 6′-fluoro-N-(4-fluorobenzyl)-4′-oxo-3′,4′-dihydro-1′H-spiro[piperidine-4,2′-quinoline]-1-carboxamide.

The invention also provides a crystalline form of the compound of 6′-fluoro-N-(4-fluorobenzyl)-4′-oxo-3′,4′-dihydro-1′H-spiro[piperidine-4,2′-quinoline]-1-carboxamide (preferably Form A), for use in a method of treatment or prevention of a disease or condition which may be treated by an AKR1C3 dependent KARS inhibitor (e.g. cancer), wherein the crystalline form of compound of 6′-fluoro-N-(4-fluorobenzyl)-4′-oxo-3′,4′-dihydro-1′H-spiro[piperidine-4,2′-quinoline]-1-carboxamide is prepared for administration with another therapeutic agent. The invention also provides another chemotherapeutic agent for use in a method of treating or preventing a disease or condition which may be treated by an AKR1C3 dependent KARS inhibitor (e.g. cancer), wherein the other therapeutic agent is prepared for administration with a crystalline form of compound of 6′-fluoro-N-(4-fluorobenzyl)-4′-oxo-3′,4′-dihydro-1′H-spiro[piperidine-4,2′-quinoline]-1-carboxamide. The invention also provides a crystalline form of 6′-fluoro-N-(4-fluorobenzyl)-4′-oxo-3′,4′-dihydro-1′H-spiro[piperidine-4,2′-quinoline]-1-carboxamide, for use in a method of treating a disease or condition which may be treated by an AKR1C3 dependent KARS inhibitor (e.g. cancer), wherein the crystalline form of 6′-fluoro-N-(4-fluorobenzyl)-4′-oxo-3′,4′-dihydro-1′H-spiro[piperidine-4,2′-quinoline]-1-carboxamide is administered with another therapeutic agent. The invention also provides another therapeutic agent for use in a method of treating a disease or condition which may be treated by an AKR1C3 dependent KARS inhibitor (e.g. cancer), wherein the other therapeutic agent is administered with a crystalline form of 6′-fluoro-N-(4-fluorobenzyl)-4′-oxo-3′,4′-dihydro-1′H-spiro[piperidine-4,2′-quinoline]-1-carboxamide.

The invention also provides the use of a crystalline form of 6′-fluoro-N-(4-fluorobenzyl)-4′-oxo-3′,4′-dihydro-1′H-spiro[piperidine-4,2′-quinoline]-1-carboxamide, for treating a disease or condition which may be treated by an AKR1C3 dependent KARS inhibitor (e.g. cancer), wherein the patient has previously (e.g. within 24 hours) been treated with another therapeutic agent. The invention also provides the use of another therapeutic agent for treating a disease or condition which may be treated by an AKR1C3 dependent KARS inhibitor (e.g. cancer), wherein the patient has previously (e.g. within 24 hours) been treated with a crystalline form of 6′-fluoro-N-(4-fluorobenzyl)-4′-oxo-3′,4′-dihydro-1′H-spiro[piperidine-4,2′-quinoline]-1-carboxamide.

### Preparation of Crystalline Form of 6′-fluoro-N-(4-fluorobenzyl)-4′-oxo-3′,4′-dihydro-1′H-spiro[piperidine-4,2′-quinoline]-1-carboxamide

Crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent or solvent mixture include, for example, evaporation of the solvent, decreasing the temperature of the solvent or solvent mixture, crystal seeding a supersaturated solvent mixture of the molecule and/or salt, freeze drying the solvent mixture, and addition of antisolvents (countersolvents) to the solvent mixture. Exemplary methods of preparing the crystalline forms described herein are set forth in detail below.

Crystals of drugs, including polymorphs, methods of preparation, and characterization of drug crystals are discussed in Solid-State Chemistry of Drugs, S. R. Byrn, R. R. Pfeiffer, and J. G. Stowell, 2^{nd} Edition, SSCI, West Lafayette, Indiana (1999).

For crystallization techniques that employ solvents, the choice of solvent or solvents is typically dependent upon one or more factors, such as solubility of the compound, crystallization technique, and vapor pressure of the solvent. Combinations of solvents may be employed, for example, the compound may be solubilized into a first solvent to afford a solution, followed by the addition of an antisolvent to decrease the solubility of the compound in the solution and to afford the formation of crystals. An antisolvent is a solvent in which the compound has low solubility.

In one method to prepare crystals, a compound is suspended and/or stirred in a suitable solvent to afford a slurry, which may be heated to promote dissolution. The term “slurry”, as used herein, means a saturated solution of the compound, which may also contain an additional amount of the compound to afford a heterogeneous mixture of the compound and a solvent at a given temperature. This may also be referred to as a suspension.

Seed crystals may be added to any crystallization mixture to promote crystallization. Seeding may be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971, 26, 369-377. In general, seeds of small size are needed to control effectively the growth of crystals in the batch. Seed of small size may be generated by sieving, milling, or micronizing of large crystals, or by micro-crystallization of solutions. Care should be taken that milling or micronizing of crystals does not result in any change in crystallinity form the desired crystal form (i.e., change to amorphous or to another polymorph).

A cooled crystallization mixture may be filtered under vacuum, and the isolated solids may be washed with a suitable solvent, such as cold recrystallization solvent, and dried under a nitrogen purge to afford the desired crystalline form. The isolated solids may be analyzed by a suitable spectroscopic or analytical technique, such as solid state nuclear magnetic resonance, differential scanning calorimetry, x-ray powder diffraction, or the like, to assure formation of the preferred crystalline form of the product. The resulting crystalline form is typically produced in an amount of greater than about 70 weight % isolated yield, preferably greater than 90 weight % isolated yield, based on the weight of the compound originally employed in the crystallization procedure. The product may be co-milled or passed through a mesh screen to delump the product, if necessary.

Alternatively, crystalline forms may be prepared directly from the reaction medium of the final process for preparing 6′-fluoro-N-(4-fluorobenzyl)-4′-oxo-3′,4′-dihydro-1′H-spiro[piperidine-4,2′-quinoline]-1-carboxamide. This may be achieved, for example, by employing in the final process step a solvent or a mixture of solvents from which 6′-fluoro-N-(4-fluorobenzyl)-4′-oxo-3′,4′-dihydro-1′H-spiro[piperidine-4,2′-quinoline]-1-carboxamide may be crystallized. In addition, crystalline forms may be obtained by distillation or solvent addition techniques.

In addition to the methods discussed briefly below, it should be understood that various analytical methods may be used for the characterization of any of the materials described herein.

The following non-limiting examples are illustrative of the disclosure.

### EXAMPLES

### Example 1: Preparation of the Anhydrous Crystalline Form A of 6′-fluoro-N-(4-fluorobenzyl)-4′-oxo-3′,4′-dihydro-1′H-spiro[piperidine-4,2′-quinoline]-1-carboxamide

Dichloromethane (DCM) (10 vol.) and Intermediate 2 (WO2021/005586) as TFA salt (1.0 eq.) were charged into a clean round bottomed flask at 23±5° C. The reaction mass was stirred for 10 minutes. Then DIPEA was charged to the reaction mass at 23±5° C. and finally 4-fluoro benzyl isocyanate was added to the reaction mass. The reaction was maintained at this temperature for 16 h. After completion of the reaction (as determined by HPLC), the reaction mass was filtered, the wet solid was washed with hexane (5 vol.), the solid sucked dry for 30 minutes then the solid was taken back in to the round bottomed flask and isopropyl alcohol (IPA) (5 vol.) added and stirred for 1 h at 23±5° C. The solid was then filtered, the wet solid was washed with hexane (5 vol.), and dried on a vacuum tray dryer (VTD) for 10 h at 50° C. Traces of DCM were detected in the final compound by 1H-NMR and the mass was re-purified with hexane and dried for longer.

The obtained product was characterised as crystalline Form A by XRPD, TGA and DSC.

In an alternative method, Form A can be prepared as follows:

An amount corresponding to 15 wt. % of Form D of 6′-fluoro-N-(4-fluorobenzyl)-4′-oxo-3′,4′-dihydro-1′H-spiro[piperidine-4,2′-quinoline]-1-carboxamide is dissolved in a mixture of acetone and water (85:15 w/w) while stirring at 50° C. The solution is filtered (clear filtration) and is then charged into a crystallizer where stirring is continued for at least 15 min at 50° C. Then, the temperature is lowered to 40° C. and a seed suspension consisting of seed crystals of Form A (micronized) dispersed in a mixture of acetone and water (40:60 w/w) is added to the system. After seeding, the mixture is stirred at 40° C. for at least 30 min before the system is cooled at a rate of 0.2° C./min to 20° C. and kept there under stirring for at least 60 min. Then, water is added continuously during 360 min so as to bring the composition of the solvent mixture to 40:60 w/w acetone:water. Finally, the system is stirred for at least 60 min after the addition of water is completed, all at 20° C. The solid is isolated from the mixture by filtration, and the filter cake washed with a mixture of acetone and water (40:60 w/w). The wet cake is unloaded and dried in the oven at 50° C. under vacuum. The material is then sieved using a hand sieve, 1 mm mesh size, to afford Form A of 6′-fluoro-N-(4-fluorobenzyl)-4′-oxo-3′,4′-dihydro-1′H-spiro[piperidine-4,2′-quinoline]-1-carboxamide as a yellow solid.

### Example 2: Preparation of Crystalline Form B of 6′-fluoro-N-(4-fluorobenzyl)-4′-oxo-3′,4′-dihydro-1′H-spiro[piperidine-4,2′-quinoline]-1-carboxamide

Form A of 6′-fluoro-N-(4-fluorobenzyl)-4′-oxo-3′,4′-dihydro-1′H-spiro[piperidine-4,2′-quinoline]-1-carboxamide was dissolved in acetone and the clear solution allowed to slowly evaporate at 25° C. The preparation was found not to be repeatable.

### Example 3: Preparation of Form C

Form A of 6′-fluoro-N-(4-fluorobenzyl)-4′-oxo-3′,4′-dihydro-1′H-spiro[piperidine-4,2′-quinoline]-1-carboxamide was dissolved in butanone and the clear solution allowed to slowly evaporate at 25° C.

The preparation was found difficult to repeat.

### Example 4: Preparation of Form D of 6′-fluoro-N-(4-fluorobenzyl)-4′-oxo-3′,4′-dihydro-1′H-spiro[piperidine-4,2′-quinoline]-1-carboxamide

Form A of 6′-fluoro-N-(4-fluorobenzyl)-4′-oxo-3′,4′-dihydro-1′H-spiro[piperidine-4,2′-quinoline]-1-carboxamide was dissolved in isopropanol and the clear solution allowed to slowly evaporate at 25° C.

In an alternative method, Form D of 6′-fluoro-N-(4-fluorobenzyl)-4′-oxo-3′,4′-dihydro-1′H-spiro[piperidine-4,2′-quinoline]-1-carboxamide can be prepared as follows:

The 4-methylbenzenesulfonate salt of 6′-fluoro-1′H-spiro[piperidine-4,2′-quinolin]-4′(3′H)-one (6.0 g, 14.8 mmol) and acetonitrile (30 mL) are charged to a reactor at 20° C. jacket temperature (JT). N,N-Diisopropylethylamine (2.3 g, 17.7 mmol) is added dropwise while holding the internal temperature (IT) at 20° C. Stirring of the resulting suspension is continued while the internal temperature is lowered to 2° C. 4-fluorobenzyl isocyanate (2.4 g, 15.9 mmol) is added dropwise in a manner that the exothermic reaction is held below 5° C. IT. Stirring at 0-5° C. IT is continued for 10 min. The resulting suspension is warmed to 20° C. IT and stirring is continued for 3 h.

H₂O (36 ml) is added portionwise over 20 min to obtain a good stirrable suspension. The resulting mixture is slowly cooled to 2° C. IT, at which stirring is continued for 1 h. The suspension is filtered cold, washed twice with H₂O and once with acetonitrile. The obtained filter cake is dried under vacuum at 55° C. JT affording crystalline Form D of 6′-fluoro-N-(4-fluorobenzyl)-4′-oxo-3′,4′-dihydro-1′H-spiro[piperidine-4,2′-quinoline]-1-carboxamide as a yellow solid (5.1 g, 13.2 mmol, 90% yield).

### Example 5: Preparation of Form E of 6′-fluoro-N-(4-fluorobenzyl)-4′-oxo-3′,4′-dihydro-1′H-spiro[piperidine-4,2′-quinoline]-1-carboxamide

Form E of 6′-fluoro-N-(4-fluorobenzyl)-4′-oxo-3′,4′-dihydro-1′H-spiro[piperidine-4,2′-quinoline]-1-carboxamide was obtained by heating Hydrate H_{A} of 6′-fluoro-N-(4-fluorobenzyl)-4′-oxo-3′,4′-dihydro-1′H-spiro[piperidine-4,2′-quinoline]-1-carboxamide to about 70° C. or exposure to 5% relative humidity for about 12 hours

### Example 6: Preparation of Hydrate Form H_{A} of 6′-fluoro-N-(4-fluorobenzyl)-4′-oxo-3′,4′-dihydro-1′H-spiro[piperidine-4,2′-quinoline]-1-carboxamide

Hydrate H_{A} of 6′-fluoro-N-(4-fluorobenzyl)-4′-oxo-3′,4′-dihydro-1′H-spiro[piperidine-4,2′-quinoline]-1-carboxamide was obtained by equilibrating the amorphous form of 6′-fluoro-N-(4-fluorobenzyl)-4′-oxo-3′,4′-dihydro-1′H-spiro[piperidine-4,2′-quinoline]-1-carboxamide in water for 4 weeks at 25° C. or by equilibrating Form A in MeOH/water=5/95 (a_{w}>0.95) at 25° C. for about 3 weeks and collecting the solid by centrifuge and drying under ambient conditions.

### Example 6: Preparation of Amorphous Form of 6′-fluoro-N-(4-fluorobenzyl)-4′-oxo-3′,4′-dihydro-1′H-spiro[piperidine-4,2′-quinoline]-1-carboxamide

Form A of 6′-fluoro-N-(4-fluorobenzyl)-4′-oxo-3′,4′-dihydro-1′H-spiro[piperidine-4,2′-quinoline]-1-carboxamide was dissolved in 1,4-dioxane. The obtained clear solution was filtered with 0.45 um

Nylon membrane. The clear solution was pre-frozen using solid carbon dioxide ethanol solution and then freeze dried under the parameters: SHLF=−24° C., SAMP=−18° C., COL=−84SEG=M, VAC=0.014 m.

### Example 7: Physico/Chemical Properties and Stability of Form a and Amorphous Form

Crystalline Form A described herein has been found to have advantageous properties.

Form A of compound of Formula (I) is the most stable form. Form A is physically stable in bulk. Form A is also chemically stable when exposed to a high percentage level of relative humidity (RH) (e.g. 75%) at increased temperature (e.g. 80° C.) and upon exposure of long-term stress conditions.

Form A is slightly hygroscopic. The maximum water uptake of Form A is 0.6% at 95% relative humidity at 25° C. Form A is stable at water activity of a_{w}=0.7 at 25° C.

Due to the slightly hygroscopic behavior, the physicochemical properties of Form A of the present invention are preserved regardless of the relative humidity of the surrounding atmosphere, which facilitates easier and more reliable manufacturing processes as well as easier storage of a pharmaceutical product containing said form A.

In addition, crystalline Form A preserves its crystal structure even when slurried (equilibrated) for prolonged time in various solvents (equilibration studies).

Form A showed an initial purity of 99.4%. In a pH stability study, 0.1% suspension/solutions of the compound of Formula (I) in buffer solutions of various pHs were exposed to 80° C. for one week. The compound of Formula (I) degraded the most in pH 1 buffer, about 5%, whereas at high pH degradation was less than 1% after 1 week at 80° C.

In the bulk, the crystalline Form A and the amorphous form of the compound of Formula (I) are chemically stable when exposed to 80° C. and 80° C. and 75% RH for one week. The amorphous form crystallized into the crystalline Modification A.

Both solid forms of the Compound of Formula (I) are also stable in mixtures with excipients. Upon light stress a slight increase in degradation products of 0.6% was noticed for the crystalline Modification A, but no degradation was observed for the amorphous form.

TABLE 1
Stability of Form A and Amorphous Form

| | Physical Form | |
|---|---|---|
| | Free form | |
| | Modification A (NX-27) | amorphous |
| Test Conditions | 99.4% | 99.3% |
| Initial purity | DP[%] | CL |

0.1% solutions or suspensions, 1 week 80° C.

| | | | | |
|---|---|---|---|---|
| pH 1.0, 0.1N HCl | 5.6 | A ↓ | — | — |
| pH 3.0, citrate buffer | 1.5 | A ↓ | — | — |
| pH 4.7, acetate buffer | 1.4 | A ↓ | — | — |
| pH 6.8, phosphate buffer | 1.2 | A ↓ | — | — |
| pH 9.0, borate buffer | 1.1 | A ↓ | — | — |
| Water | 1.2 | A ↓ | — | — |
| Ethanol | 16.6 | A * | — | — |
| Acetonitrile | 1.2 | A * | — | — |
| MEK | 7.6 | A * | — | — |
| Dioxane | 56.8 | A * | — | — |
| NMP | 14.3 | A * | — | — |

Solid state, 1 week 80° C., tight container

| | | | | |
|---|---|---|---|---|
| Bulk (HPLC) | 0.6 | — | 0.7 | — |
| Bulk (XRPD) | No change | — | NX Mod A | — |

2 weeks 50° C., tight container

| | | | | |
|---|---|---|---|---|
| 1% in mixture 1 | 0.4 | — | 0.8 | — |
| 1% in mixture 2 | 0.4 | — | 0.8 | — |
| 1% in mixture 3 | 0.4 | — | 0.8 | — |

Solid state, 1 week 80° C./75% RH

| | | | | |
|---|---|---|---|---|
| Bulk (HPLC) | 0.7 | — | 0.7 | — |
| Bulk (XRPD) | No change | — | NX Mod A | — |

2 weeks 50° C./75% RH

| | | | | |
|---|---|---|---|---|
| 1% in mixture 1 | 0.4 | — | 0.8 | — |
| 1% in mixture 2 | 0.4 | — | 0.8 | — |
| 1% in mixture 3 | 0.4 | — | 0.8 | — |

Xenon light (1200 kLuxh)

| | | | | |
|---|---|---|---|---|
| Bulk (HPLC) | 1.2 | B | 0.7 | A |
| Bulk (XRPD) | No change | | No change | |

Degradation products (DP) and color (CL)

| | | | |
|---|---|---|---|
| ↓ | Suspension | * | Clear solution after stress test |
| — | Test not performed | A | No change of color |
| B | Slight discoloration | C | Medium discoloration |
| D | Strong discoloration | | |

DPs are analyzed by HPLC (method see Appendix 2) They are calculated as area-% products.
Compositions of the excipient mixtures [mass-%]

| | | |
|---|---|---|
| No. | Material | % w/w |

Mixture 1 - Dry Blend

| | | |
|---|---|---|
| 1 | Mannitol powder | 47.4 |
| 2 | Starch 1500 | 36.8 |
| 3 | Sodium starch glycolate | 6.3 |
| 4 | Hydroxypropyl cellulose 100 cps | 4.2 |
| 5 | Sodium stearylfumarate | 3.2 |
| 6 | Talc | 2.1 |

Mixture 2 - Wet granulation

| | | |
|---|---|---|
| 1 | MCC PH101 | 29.5 |
| 2 | Lactose monohydrate | 56.8 |
| 3 | Croscarmellose sodium | 6.3 |
| 4 | Hydroxypropyl methylcellulose 3 cps | 4.2 |
| 5 | Magnesium stearate | 1.1 |
| 6 | Aerosil 200 | 2.1 |

Mixture 3 - Roller compaction

| | | |
|---|---|---|
| 1 | MCC PH102 | 55.8 |
| 2 | Calcium hydrogen phosphate | 22.1 |
| 3 | L-Hydroxypropyl cellulose | 10.5 |
| 4 | Hydroxypropyl cellulose 100 cps | 5.3 |
| 5 | Magnesium stearate | 3.2 |
| 6 | Aerosil 200 | 3.2 |

### Example 8: High-Resolution Powder X-Ray Diffraction

A sample of material from Example 1, was placed into a silicon specimen holder for reflection measurements and the holder was placed in the diffractometer. XRPD patterns were collected at room temperature (about 296 K) on a Bruker D8 Advance system equipped with LynxEye solid state detector in reflection mode. The radiation used for collecting the data was CuKα (λ=0.15418 nm). Diffraction data were collected in the range 2-40°2θ and peaks evaluated. The obtained spectrum is shown in FIG. 1 and the peaks are listed in Table 1. The procedure was repeated with the material obtained from Examples 2-6.

TABLE 1
X-ray powder diffraction data for anhydrous crystalline Form A

| 2θ [°] | d value [Å] | Intensity [%] |
|---|---|---|
| 9.57 | 9.239 | 100 |
| 10.45 | 8.458 | 19 |
| 13.35 | 6.629 | 19 |
| 15.74 | 5.625 | 16 |
| 17.11 | 5.178 | 32 |
| 19.17 | 4.626 | 79 |
| 21.01 | 4.225 | 40 |
| 22.42 | 3.962 | 11 |
| 27.25 | 3.270 | 11 |
| 30.42 | 2.936 | 21 |
| 31.69 | 2.821 | 13 |

TABLE 2
X-ray powder diffraction data for anhydrous crystalline Form B

| 2θ [°] | d value [Å] | Intensity [%] |
|---|---|---|
| 9.70 | 9.111 | 11 |
| 13.61 | 6.501 | 9 |
| 15.32 | 5.779 | 100 |
| 16.48 | 5.374 | 20 |
| 18.80 | 4.716 | 16 |
| 20.08 | 4.419 | 17 |

TABLE 3
X-ray powder diffraction data for anhydrous crystalline Form C

| 2θ [°] | d value [Å] | Intensity [%] |
|---|---|---|
| 7.12 | 12.406 | 3 |
| 11.10 | 7.961 | 8 |
| 14.23 | 6.219 | 5 |
| 17.25 | 5.137 | 5 |
| 20.35 | 4.361 | 13 |
| 21.40 | 4.150 | 6 |
| 22.26 | 3.990 | 100 |
| 29.28 | 3.047 | 12 |

TABLE 4
X-ray powder diffraction data for anhydrous crystalline Form D

| 2θ [°] | d value [Å] | Intensity [%] |
|---|---|---|
| 8.18 | 10.798 | 31 |
| 10.63 | 8.313 | 15 |
| 13.53 | 6.540 | 45 |
| 13.82 | 6.404 | 13 |
| 17.14 | 5.169 | 76 |
| 17.47 | 5.073 | 39 |
| 20.28 | 4.375 | 71 |
| 21.34 | 4.161 | 19 |
| 21.84 | 4.067 | 17 |
| 24.69 | 3.602 | 100 |
| 28.49 | 3.131 | 57 |

TABLE 5
X-ray powder diffraction data for anhydrous crystalline Form E

| 2θ [°] | d value [Å] | Intensity [%] |
|---|---|---|
| 11.14 | 7.933 | 10 |
| 18.15 | 4.885 | 7 |
| 20.52 | 4.326 | 11 |
| 22.33 | 3.978 | 100 |
| 29.04 | 3.073 | 18 |

TABLE 6
X-ray powder diffraction data for hydrate Form H_{A}

| 2θ [°] | d value [Å] | Intensity [%] |
|---|---|---|
| 9.97 | 8.861 | 25 |
| 14.69 | 6.026 | 43 |
| 18.67 | 4.749 | 22 |
| 20.00 | 4.436 | 100 |
| 21.46 | 4.137 | 26 |
| 24.18 | 3.678 | 11 |

### Example 8: Differential Scanning Calorimetry (DSC)

DSC thermograms were recorded with a TA Discovery DSC instrument using heat flux compensation (TA Instruments, USA). The TA Discovery instrument was calibrated for temperature and enthalpy according to the manufacturer's instructions using certified reference substances like indium. About 2 mg of sample material was sealed in a standard pin-holed aluminum pan and heated in the DSC from 0° C. to 300° C., at a heating rate of 10° C./min. Dry nitrogen gas, at a flow rate of 50 ml/min was used to purge the DSC equipment during the measurement. The obtained graphs are shown in the Figures.

### Example 9: Thermogravimetric Analysis (TGA)

Thermogravimetric analysis was performed using a TA Discovery TGA instrument (TA Instruments, USA). About 2-10 mg of sample material was sealed in a standard aluminum pan with pierced lid and heated in the TGA from 30° C. to 250° C., at a heating rate of 10° C./min. Dry N₂ gas, at a flow rate of 20 ml/min was used to purge the TGA equipment during the measurement. The obtained graphs are shown in the Figures.

The experimental investigation concludes that 6′-fluoro-N-(4-fluorobenzyl)-4′-oxo-3′,4′-dihydro-1′H-spiro[piperidine-4,2′-quinoline]-1-carboxamide shows polymorphic behavior.

Form A is an anhydrous crystalline form with a melting onset of about 208° C. and melting enthalpy of about 111-119 J/g. The melting onset temperature does not significantly depend on the heating rate as demonstrated by DSC performed using different heating rates. Form A is only slightly hygroscopic and uptakes maximal 0.6% of water at 95% relative humidity at 25° C.

The weight loss by TGA is about 0.3% in the range of about 24-200° C.

In addition to the crystalline Form A, several other assigned solid forms were identified based on those each exhibiting a different XRPD spectrum as detailed below.

Form B was obtained by evaporation crystallization in acetone. Form B shows a different XRPD pattern to Form A. The DSC shows a melting onset of about 184° C. followed by recrystallization into Form A with melting onset of about 206° C. TGA analysis of Form B showed loss on drying of 2.8% between about 28° C. and 190° C. However, it was not possible to reproduce Form B and further characterize it.

Form C was obtained by slow evaporation in butanone. However, it is difficult to reproduce and only Form A was obtained in crystallization trials. Form C transforms upon heating at about 150° C. into Form A. The DSC of Form C shows an endothermic transition into Form A at about 152° C. to 159° C. followed by melting of Form A at 207°−208° C. The endothermic transition suggests an enantiotropic relationship of Form C and Form A. Based on competitive slurry experiments, which showed conversion of Form C into Form A at 25° C., a transition temperature will be below 25° C.

Form D was obtained by slow evaporation in isopropanol. Form D shows a different XRPD pattern to the other Forms which suggests that Form D is a pure crystalline phase. Form D started to convert into Form A above about 190° C. The DSC of Form D shows a small endothermic event at about 70° C. followed by melting onset of 204° C. and an enthalpy of 106 J/g. The DSC data suggest a monotropic relationship of Form A and Form D. Competitive slurry experiment shows that Form D will convert into Form A at 25° C.

Form E was obtained by heating Hydrate H_{A} to about 70° C. or exposure to 5% relative humidity for about 12 hours. Form E transforms into Form A upon heating to 150° C. The DSC of Form E shows a small exothermic event starting at about 115° C. followed by melting at 207° C., the melting point of Form A.

The obtained data suggests that the forms Forms B, C, D and E are metastable crystalline forms in respect to Form A.

Hydrate H_{A} was obtained by equilibrating the amorphous form in water for 4 weeks at 25° C. or by equilibrating Form A in MeOH/water=5/95 (a_{w}>0.95) at 25° C. for about 3 weeks and collecting the solid by centrifuge and drying under ambient conditions. Hydrate H_{A} is a monohydrate with a calculated water content of 4.5%.

Hydrate H_{A} remains stable in humidity controlled XRPD when the relative humidity is decreased from 40% RH to 5% RH at 25° C. After exposure of the Hydrate H_{A} for about 12 hours to 5% RH the XRPD pattern changed to Form E.

Hydrate H_{A} also transforms into Form E at about 70° C., which then transforms into Form A at about 150° C.

The amorphous form was obtained by dissolving Form A in 1,4-dioxane and freeze drying the resultant solution.

The slurry experiments of Form A and Hydrate H_{A} in media with different water activities suggest that at 25° C. Form A is stable at water activities of a_{w}=0.7 and below, whereas the hydrate is stable at a_{w}=0.9 and above. At 40° C. Form A is stable at water activities of a_{w}=0.8 and below, whereas the hydrate is stable at a_{w}>0.95.

1. A crystalline form of the compound 6′-fluoro-N-(4-fluorobenzyl)-4′-oxo-3′,4′-dihydro-1′H-spiro[piperidine-4,2′-quinoline]-1-carboxamide.

2. The crystalline form according to claim 1 of the compound 6′-fluoro-N-(4-fluorobenzyl)-4′-oxo-3′,4′-dihydro-1′H-spiro[piperidine-4,2′-quinoline]-1-carboxamide, characterized by an x-ray powder diffraction pattern comprising a representative peak, in terms of °2θ, at 9.6±0.2°2θ when measured at a temperature of about 25° C.

3. A crystalline form of the compound 6′-fluoro-N-(4-fluorobenzyl)-4′-oxo-3′,4′-dihydro-1′H-spiro[piperidine-4,2′-quinoline]-1-carboxamide, characterized by an x-ray powder diffraction pattern comprising one or more representative peaks in terms of 2 θ selected from the group consisting of 9.6±0.2°2θ, 10.5±0.2°2θ, 13.4±0.2°2θ, 15.7±0.2°2θ, 17.1±0.2°2θ, 19.2±0.2°2θ, 21.0±0.2°2θ, 22.4±0.2°2θ, 27.3±0.2°2θ, 30.4±0.2°2θ and 31.7±0.2°2θ, measured at a temperature of about 25° C.

4. The crystalline form according to claim 1 having an x-ray diffraction spectrum substantially the same as the x-ray powder diffraction spectrum shown in FIG. 1.

5. The crystalline form of claim 1, characterized by a differential thermogravimetric profile measured by Differential Scanning calorimetry (DSC) with a heating rate of 10° C./min, comprising a single endothermic peak starting at about 208° C.

6. The crystalline form according to claim 1 having a differential scanning calorimetry (DSC) thermogram substantially the same as that shown in FIG. 2.

7. The crystalline form of claim 1, having a decomposition point greater than 200° C. and a weight loss on drying of about 0.3% in the range of 24-200° C., as determined by thermogravimetric analysis.

8. The crystalline form according to claim 1 having a thermogravimetric analysis (TGA) diagram substantially the same as that shown in FIG. 3.

9. A pharmaceutical composition comprising a crystalline form of claim 1, and a pharmaceutically acceptable carrier.

10. (canceled)

11. (canceled)

12. A method for the treatment of a disorder ameliorated by an AKR1C3 dependent KARS inhibitor, comprising administering to a patient in need of such treatment an effective amount of a substantially phase pure crystalline form of 6′-fluoro-N-(4-fluorobenzyl)-4′-oxo-3′,4′-dihydro-1′H-spiro[piperidine-4,2′-quinoline]-1-carboxamide according to claim 1.

13. The method of claim 12, wherein the disorder ameliorated by an AKR1C3 dependent KARS inhibitor is selected from non-small cell lung cancer (NSCLC), liver cancer, head and neck cancer, esophageal cancer, uterine cancer, breast cancer, bladder cancer, cervical cancer, colorectal cancer, kidney cancer, melanoma, stomach, castration-resistant prostate cancer (CRPC), T-cell acute lymphoblastic leukemia (T-ALL), acute myeloid leukemia (AML), and myelodysplastic syndrome (MDS).

14. The method according to claim 12, wherein the disorder is non-small cell lung cancer (NSCLC).

15. A process for making crystalline Form A of 6′-fluoro-N-(4-fluorobenzyl)-4′-oxo-3′,4′-dihydro-1′H-spiro[piperidine-4,2′-quinoline]-1-carboxamide, said process which comprising the steps of:
a) Reacting 6′-fluoro-1′H-spiro[piperidine-4,2′-quinolin]-4′(3′H)-one with 4-fluorobenzyl isocyanate in a chlorinated solvent, optionally in the presence of a base; and
b) Isolating the formed solid.

16. a process for making crystalline Form A of 6′-fluoro-N-(4-fluorobenzyl)-4′-oxo-3′,4′-dihydro-1′H-spiro[piperidine-4,2′-quinoline]-1-carboxamide, said process comprising the steps of:
a) Dissolving an amount of 6′-fluoro-N-(4-fluorobenzyl)-4′-oxo-3′,4′-dihydro-1′H-spiro[piperidine-4,2′-quinoline]-1-carboxamide in a solvent;
b) Adding seed crystals of Form A of 6′-fluoro-N-(4-fluorobenzyl)-4′-oxo-3′,4′-dihydro-1′H-spiro[piperidine-4,2′-quinoline]-1-carboxamide; and
c) Isolating the formed solid.
